(19) United States
(12) Patent Application Publication
(10) Pub. No.: US 2026/0053809 A1
(43) Pub. Date: Feb. 26, 2026

(54) MOLECULAR THERAPEUTIC STRATEGY COMBINING IDELALISIB AND SRPIN340 TO TREAT ADVANCE SOLID TUMORS

(71) Applicant: UNIVERSITY OF MARYLAND, EASTERN SHORE, Princess Anne, MD (US)

(72) Inventors: Bi-Dar Wang, Salisbury, MD (US); Siyoung Ha, Salisbury, MD (US)

(21) Appl. No.: 19/301,452

(22) Filed: Aug. 15, 2025

Related U.S. Application Data
(60) Provisional application No. 63/685,074, filed on Aug. 20, 2024.

(51) Int. Cl. A61K 31/52; A61K 31/404; A61K 31/4545; A61K 31/496; A61K 38/46; A61P 35/00

(52) U.S. Cl. CPC A61K 31/52; A61K 31/404; A61K 31/4545; A61K 31/496; A61K 38/465; A61P 35/00; C12Y 301/03067

(57) ABSTRACT

PI3Kδ implicates hematologic cancers and solid tumors. Alternative splicing is a post-transcriptional process for acquiring proteomic diversity in eukaryotic cells. Emerging evidence highlights the involvement of aberrant mRNA splicing in cancer development/progression. PI3Kδ-L and PI3Kδ-S are overexpressed in advanced solid tumors, such as prostate, breast, colon, lung and pancreatic cancers. Differential PI3Kδ and PI3Kδ-S expression profiles were identified in a panel of solid tumor cells. PI3Kδ inhibitor Idelalisib and SRPK1/2 inhibitor SRPIN340 were employed to assess their efficacies on inhibiting the PI3Kδ-expressing solid tumors. Idelalisib effectively inhibits PI3Kδ-L and its downstream signaling. Idelalisib fails to inhibit PI3Kδ-S activity and its downstream signaling. SRPIN340 reverses the aberrant mRNA splicing, thereby inhibiting the downstream AKT/mTOR signaling. In vitro functional assays further demonstrate that a combination of Idelalisib and SRPIN340 achieve a synergistic drug effect, with drastically reduced cell viabilities/growths of tumor spheroids, in inhibiting the advanced tumor cells.

### CROSS REFERENCE TO RELATED APPLICATIONS

This application claims priority under 35 U.S.C. § 119(e) to provisional patent application U.S. Ser. No. 63/685,074, filed Aug. 20, 2024. The provisional patent application is hereby incorporated by reference in its entirety herein, including without limitation: the specification, claims, and abstract, as well as any figures, tables, appendices, or drawings thereof.

### STATEMENT REGARDING FEDERALLY SPONSORED RESEARCH OR DEVELOPMENT

This invention was made with government support under GM127256 awarded by the National Institutes of Health. The government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Aug. 14, 2025, is named “P14917US01_SequenceListing.xml” and is 5,570 bytes in size.

### TECHNICAL FIELD

The present disclosure relates generally to molecular therapeutic strategy combining Idelalisib and SRPK1/2 inhibitor SRPIN340. More particularly, but not exclusively, the present disclosure relates to the use of the aberrant PI3Kδ-S (or PIK3CD-S splice variant) as a precision biomarker and to further develop PI3Kδ-S as a novel drug target for the PCa and other endocrine/solid tumors.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Phosphatidylinositol-3-kinase (PI3K)/AKT signaling pathway is upregulated in a broad spectrum of cancers. Among the class I PI3Ks (PI3Kα/β/δ isoforms), PI3Kδ has been implicated in hematologic cancers and solid tumors. Alternative splicing is a post-transcriptional process for acquiring proteomic diversity in eukaryotic cells. Emerging evidence has highlighted the involvement of aberrant mRNA splicing in cancer development/progression.

PI3K/AKT signaling regulates important cellular events, including cell proliferation, survival, mobility, metabolism and angiogenesis. Hyperactivation of PI3K/AKT signaling is involved in the development of cancers and treatment resistance. Therefore, targeting the modulators/components of this pathway has attracted attentions as promising therapeutic options. PI3K is a lipid and protein kinase that converts phosphatidylinositol 4,5-bisphosphate (PIP2) to phosphatidylinositol 3,4,5-trisphosphate (PIP3). Class I PI3Ks are heterodimers that consist of a catalytic subunit (PI3Kα, PI3 Kβ, or PI3Kδ) and a regulatory subunit (p85α, p85β, or p55γ). PI3Kα and PI3Kβ are found to be ubiquitously expressed, while PI3Kδ is frequently enriched in leukocytes. Phosphatase and tensin homolog (PTEN) is a lipid phosphatase that dephosphorylates/antagonizes the PI3K product, PIP3. PTEN negatively regulates PI3K and inhibits PI3K/AKT signaling, consequently suppressing cell growth, proliferation and migration in cancers. Conversely, loss of PTEN causes an accumulation of PIP3 and lead to a constitutive activation of PI3K/AKT pathway, leading to cell transformation and development of various cancers.

Overexpression of PI3Kδ is frequently involved in hematologic malignancies such as chronic lymphocytic leukemia (CLL), multiple myeloma (MM), lymphoma, non-Hodgkin's lymphoma (NHL), acute myeloid leukemia (AML), and acute promyelocytic leukemia (APL). Emerging evidence has suggested that PI3Kδ is also overexpressed in a wide array of solid tumors, including PCa, breast cancer, ovarian cancer, colon cancer, lung cancer, liver cancer, glioblastoma, and neuroblastoma. Specifically, PI3Kδ is overexpressed in colorectal cancer (CRC), and ectopic expression of PIK3CD (gene encoding PI3Kδ) significantly activate the AKT signaling and promotes CRC cell growth, migration and invasion in vitro and tumor growth in vivo. Upregulation of PI3Kδ was found in hepatocellular carcinoma (HCC), and the higher expression levels of PI3Kδ were correlated with early relapse and poorer survival rates in HCC patients. PI3Kδ has also been shown to regulate the biochemical and bioinformatic properties of breast cancers. For instance, upregulation of PI3Kδ promotes the cell migration, invasion, and spheroid growth of the breast cancer cell lines. Overexpression of PI3Kδ was also detected in primary neuroblastomas and cell lines. In contrast, siRNA knockdown of PIK3CD resulted in reduced cell proliferation and enhanced cell apoptosis in neuroblastoma cells. In addition, PI3Kδ is highly expressed in various glioblastomas. SiRNA knockdown of PIK3CD resulted in a significant reduction of cell invasion and migration in glioblastoma cell lines. Take all together, these studies have suggested PI3Kδ as a potential biomarker and drug target for solid tumors.

Alternative splicing is a post-transcriptional process to generate alternative mRNA transcripts that encodes structurally and sometimes also functionally distinct protein isoforms. Genomic analysis such as deep sequencing studies have further revealed that >95% of human genes are undergone alternative splicing at the pre-mRNA level. Emerging evidence has revealed the critical function roles of alternative splicing in cancer development and progression. See e.g., Calderon-Aparicio et al., “Prostate cancer: Alternatively spliced mRNA transcripts in tumor progression and their uses as therapeutic targets”, Int J Biochem Cell Biol, 2021. 141: p. 106096; Wang et al. “Aberrant RNA Splicing in Cancer and Drug Resistance”, Cancers (Basel), 2018. 10 (11); both of which are hereby incorporated by reference in their entireties herein. Previously, the genomic data have revealed >2,500 alternative splicing events between African American (AA) vs. European American (EA) prostate cancer (PCa) specimens. Several oncogenes and tumor suppressor genes (i.e. PIK3CD, FGFR3, TSC2, ITGA4, MET, NF1, BAK1, and RASGRP2) were identified with differential splicing patterns in AA vs. EA PCa. See e.g., Wang et al., “Alternative splicing promotes tumor aggressiveness and drug resistance in African American prostate cancer”, Nat Commun, 2017. 8: p. 15921, which is hereby incorporated by reference in its entirety herein. Notably, PIK3CD-S splice variant exhibits more oncogenic properties than the full-length PIK3CD-L. Also, PI3Kδ-S (encoded from PIK3CD-S) demonstrated a higher kinase activity, more proliferative/invasive capacities, enhanced activation of AKT/mTOR signaling, when compared to the full-length PI3Kδ-L. Additionally, molecular modeling, ATP-competitive assays and cell-free kinase assays have further suggested/confirmed that PI3Kδ-S (lacking critical drug binding sites in its catalytic domain due to the exon 20 skipping) is more resistance to the PI3K/PI3Kδ inhibitors, when compared to PI3Kδ-L. See e.g., Ha et al., “Molecular Insight into Drug Resistance Mechanism Conferred by Aberrant PIK3CD Splice Variant in African American Prostate Cancer” Cancers (Basel), 2023. 15(4), which is hereby incorporated by reference in its entirety herein.

Thus, there exists a need in the art to evaluate the potential of utilizing the aberrant PI3Kδ-S (or PIK3CD-S splice variant) as a precision biomarker and to further develop PI3Kδ-S as a novel drug target for the PCa and other endocrine/solid tumors.

### SUMMARY

PI3K/AKT signaling pathway is upregulated in a broad spectrum of cancers. Among the class I PI3Ks (PI3Kα/β/δ isoforms), PI3Kδ has been implicated in hematologic cancers and solid tumors. Alternative splicing is a post-transcriptional process for acquiring proteomic diversity in eukaryotic cells. Emerging evidence has highlighted the involvement of aberrant mRNA splicing in cancer development/progression. Previously, the present inventors had revealed that PIK3CD-S is an oncogenic splice variant that promotes tumor aggressiveness and drug resistance in prostate cancer (PCa). To further evaluate the potentials of utilizing PI3Kδ-S (encoded from PIK3CD-S) as a cancer biomarker and/or drug target, comprehensive analyses were performed in a series of patient samples and cell lines derived from endocrine/solid tumors. Specifically, IHC, immunofluorescence, western blot and RT-qPCR assay results have demonstrated that PI3Kδ isoforms were highly expressed in endocrine/solid tumor patient specimens and cell lines.

Differential PIK3CD-S/PIK3CD-L expression profiles were identified in a panel of endocrine/solid tumor cells. SiRNA knockdown of PIK3CD-L or PIK3CD-S differentially inhibits AKT/mTOR signaling in PCa, breast, colon and lung cancer cell lines.

SiRNA knockdown of PTEN increased PI3Kδ levels and activated AKT/mTOR signaling, while overexpression of PTEN reduced PI3Kδ levels and inhibited AKT/mTOR signaling in cancer cells. Intriguingly, PI3Kδ-S levels remained unchanged upon either siRNA knockdown or overexpression of PTEN.

Taken together, these results suggest that PTEN negatively regulates PI3Kδ-L and its downstream AKT/mTOR signaling, while PI3Kδ-S promotes AKT/mTOR signaling without regulation by PTEN.

PI3Kδ inhibitor Idelalisib and SRPK1/2 inhibitor SRPIN340 were employed to assess their efficacies on inhibiting the PI3Kδ-expressing endocrine/solid tumors.

Idelalisib effectively inhibits PI3Kδ-L (but not PI3Kδ-S) mediated AKT/mTOR signaling. In contrast, SRPIN340 reversed the aberrant mRNA splicing, thereby inhibiting AKT/mTOR signaling.

In vitro functional assays further demonstrate that a combination of Idelalisib and SRPIN340 achieved a synergistic drug effect (with drastically reduced cell viabilities/growths of tumor spheroids) in inhibiting the advanced tumor cells.

In summary, the present disclosure shows promising potential of utilizing PI3Kδ-S (an oncogenic isoform conferring drug resistance and exempt from PTEN regulation) as a prognostic biomarker and drug target in advanced endocrine cancers.

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to examine the expression profiles of PI3Kδ-S/PI3Kδ-L (or PIK3CD-S/PIK3CD-L) in a panel of patient samples and cell lines derived from endocrine/solid tumors, including PCa, breast, pancreatic, colon and lung cancers. For example, immunohistochemistry (IHC), immunofluorescence, RT-PCR and western blot assays can be performed.

It is still yet a further object, feature, and/or advantage of the present disclosure to investigate the expression correlation and regulatory roles between PTEN and PI3Kδ or PI3Kδ-S. For example, IHC and western blot analyses can be used.

It is still yet a further object, feature, and/or advantage of the present disclosure to investigate the inhibitory effects on AKT/mTOR signaling in endocrine/solid tumor cells. For example, siRNA knockdown of PIK3CD-L and/or PIK3CD-S followed by western blot assays can be conducted.

It is still yet a further object, feature, and/or advantage of the present disclosure to evaluate the therapeutic effects of direct targeting PI3Kδ and/or modulating splicing mechanism on inhibiting the endocrine/solid tumors expressing PIK3CD-L/PIK3CD-S. For example, Idelalisib and SRPK1/2 inhibitor SRPIN340 can be used as single agents or in combination.

It is still yet a further object, feature, and/or advantage of the present disclosure to provide a systemic analysis of PI3Kδ-L and PI3Kδ-S expression profiles in different endocrine/solid tumor specimens and cell lines. By applying approaches of molecular/cellular biology, biochemistry, histology and in vitro functional assays, the present disclosure implicates a potential of using PI3Kδ-S as a prognostic biomarker and/or therapeutic target for endocrine cancers.

According to some aspects of the present disclosure, a therapeutic comprises a phosphoinositide 3-kinase (PI3K) inhibitor; and a serine-arginine-rich protein kinase (SRPK) inhibitor.

According to some additional aspects of the present disclosure, the PI3K inhibitor comprises a phosphoinositide 3-kinase-8 (PI3Kδ) inhibitor. The PI3Kδ inhibitor can comprise idelalisib.

According to some additional aspects of the present disclosure, the SRPK inhibitor comprises SRPIN340.

According to some additional aspects of the present disclosure, the SRPK inhibitor comprises a SRPK1/2 inhibitor. The SRPK1/2 inhibitor can comprise SRPKIN-1 or SPHINX31.

According to some additional aspects of the present disclosure, the therapeutic further comprises an additional tumor suppressor. The additional tumor suppressor can comprise phosphatase and tensin homolog (PTEN).

According to some other aspects of the present disclosure, a method comprises reversing an aberrant splicing in tumor cells with a serine-arginine-rich protein kinase (SRPK) inhibitor.

According to some additional aspects of the present disclosure, the method further comprises, prior to the reversing step, using a phosphoinositide 3-kinase-8 (PI3Kδ) inhibitor to target tumor cells.

According to some additional aspects of the present disclosure, the method further comprises further sensitizing the tumors cells to the PI3Kδ inhibitor.

According to some additional aspects of the present disclosure, the method further comprises converting PI3Kδ-S to PI3Kδ-L.

According to some additional aspects of the present disclosure, the method further comprises inhibiting PI3Kδ-S synthesis.

According to some additional aspects of the present disclosure, the method further comprises RNA splice switching.

According to some additional aspects of the present disclosure, the method further comprises utilizing phosphatase and tensin homolog (PTEN) to further regulate PI3K/AKT/mTOR signaling.

According to some additional aspects of the present disclosure, a type of the tumor cells is selected from the group consisting of: PCa, breast, endocrine, pancreatic, colon, and lung cancers.

According to some other aspects of the present disclosure, a method of biological identification comprises identifying whether there is presence of a precision biomarker comprising PI3Kδ-L or PI3Kδ-S.

According to some additional aspects of the present disclosure, the method further comprises diagnosing cancer or predicting an outcome of a disease based upon the presence of the precision biomarker.

According to some additional aspects of the present disclosure, the method further comprises measuring an aspect of the AKT/mTOR signaling pathway after confirming the presence of the precision biomarker.

According to some additional aspects of the present disclosure, the method further comprises measuring a biological state or a condition in a solid tumor.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIGS. 1A-1D show expression levels of PI3Kδ and PI3Kδ-S splice isoforms, at protein and RNA levels, in a panel of PCa patient samples and cell lines. FIG. 1A shows IHC staining assays showing the expression levels of AMACR, PI3Kδ and PI3Kδ-S in six PCa specimens (#1 to #6) on the TMAs. N: normal prostate tissues. Scale bar: 20 mm. FIG. 1B shows quantification of PI3Kδ and PI3Kδ-S intensities from the IHC images of the PCa patients and normal controls on TMAs. Significantly different (***p-value<0.001) PI3Kδ intensities between PCa specimens and normal prostate tissues. The p-values were determined based on the two-tailed student t-test. Data values represent mean±SEM. FIG. 1C shows RT-PCR assays to examine the expression profiles of full-length PIK3CD-L and PIK3CD-S splice variant in PCa cell lines 22Rv1, PC-3, LNCaP, MDA PCa 2b, DU-145, and C4-2B. The PIK3CD-S/PIK3CD-L (S/L) ratios were determined as described in Methods. FIG. 1D includes immunofluorescence assays results showing the expression of representative PI3Kδ (green fluorescence) and PI3Kδ-S (red fluorescence) signals in PCa (22Rv1, PC-3, LNCaP, DU-145, C4-2B, and MDA PCa 2b) cells. Nuclei were counterstained with DAPI (blue), and the Merged images were obtained by overlaying PI3Kδ, PI3Kδ-S with DAPI signals. Scale bar: 5 mm.

FIGS. 2A-2D show PI3Kδ-L and PI3Kδ-S splice isoform are generally overexpressed in solid tumors, including endocrine cancers. FIG. 2A shows representative IHC staining images for breast, lung, colon, prostate and pancreatic cancers. A subset of PI3Kδ-positive patient samples derived from the endocrine/solid tumors expressed PI3Kδ-S splice isoform (right panel). Scale bar: 20 mm. FIG. 2B shows quantification of PI3Kδ and PI3Kδ-S levels from the IHC staining images of breast, prostate, pancreatic, lung, and colon cancers specimens on the TMAs. The mean and SEM values were shown on the dot plots. FIG. 2C shows RT-PCR assays to examine the expression profiles of PIK3CD-L and PIK3CD-S splice variant in colon (HT-29 and SW620), lung (A549 and H1299), and breast (MDA MB 231 and MCF-7) cancer cell lines. The PIK3CD-S/PIK3CD-L (S/L) ratios were determined as described in Methods. FIG. 2D shows immunofluorescence assays showing the expression levels of PI3Kδ (green fluorescence) and PI3Kδ-S (red fluorescence) in colon (HT29 and SW620), lung (A549 and H1299), and breast (MDA MB 231 and MCF-7) cancer cells. Nuclei were counterstained with DAPI (blue), and Merge images were generated by overlaying PI3Kδ and PI3Kδ-S with DAPI signals. Scale bar: 5 mm. ns: not significant. *p-value<0.05, **p-value<0.01, and ****p-value<0.0001 were determined based on ANOVA with Dunnett's post hoc test.

FIGS. 3A-3B show higher PI3Kδ-S levels are correlated to higher Gleason scores. FIG. 3A shows normalized PI3Kδ and FIG. 3B shows PI3Kδ-S intensities in PCa specimens with Gleason scores of 2+3, 3+3, 4+3, and 5+4. The PI3Kδ and PI3Kδ-S intensities were measured based on IHC images. Significantly different PI3Kδ-S intensities (*p<0.05 and **p<0.01, one way ANOVA with Tukey's post hoc test) between patients with higher Gleason scores (4+3 or 5+4) and low Gleason scores (2+3). Data values were based on mean±SEM.

FIGS. 4A-4E show IHC staining results revealed PI3Kδ-S as a potential precision prognostic biomarker in endocrine and solid tumors. Representative IHC images of PI3Kδ and PI3Kδ-S levels in different pathological grades of prostate cancer (FIG. 4A), breast cancer (FIG. 4B), pancreatic cancer (FIG. 4C), colon cancer (FIG. 4D), and lung cancer specimens (FIG. 4E). Different Gleason Scores were represented as 2+3, 3+3, 4+3, and 5+4. G1: grade 1; G2: grade 2; G3: grade 3. The intensities/scores of PI3Kδ and PI3Kδ-S were labeled on the top left corners of IHC images.

FIGS. 5A-5B show siRNA knockdown of PIK3CD splice variants followed by cell viability assays. FIG. 5A shows RT-PCR assays to verify the efficiencies of siRNA knockdown of total PIK3CD splice variants (siPIK3CD), PIK3CD-L (siPIK3CD-L) and PIK3CD-S (siPIK3CD-S). EIF1AX was used as endogenous control for RT PCR assay. FIG. 5B shows cell viabilities after the cancer cells were transfected with siRNAs for twenty-four hours (24 h). The data were presented as mean±SD, from 3-4 independent experimental repeats.

FIGS. 6A-6C show inhibitory effects of AKT/mTOR signaling upon siRNA knockdown of total PIK3CD (PIK3CD-L and its splice variants), PIK3CD-L or PIK3CD-S in PCa, colon, lung and breast cancer cell lines. FIG. 6A show western blot analysis of pAKT, AKT, pS6 and S6 in the cancer cells transfected with nonsense siRNA (NS), siPIK3CD, siPIK3CD-L or siPIK3CD-S. β-actin was used as an endogenous control for the western blot analysis of pAKT, AKT, pS6 and S6. FIG. 6B shows quantification of pAKT, AKT, pS6 and S6 levels (of all experimental groups in A, normalized to NS controls) from 3-4 independent western blot results. FIG. 6C shows BrdU-labeling cell proliferation assays in PCa cells transfected with NS, siPIK3CD, siPIK3CD-L or siPIK3CD-S. All the bar graphs in (FIG. 6B, FIG. 6C) were presented as mean±SD, and the significances (*p-value<0.05, in siRNA knockdown vs. NS) were calculated based on one-way ANOVA with Dunnett's post hoc tests.

FIGS. 7A-7C show PTEN negatively regulates expression levels of total PI3Kδ, but not PI3Kδ-S splice isoform. FIG. 7A shows representative IHC staining images showing three categories of PTEN/PI3Kδ expression profiles/patterns in PCa patient specimens. Specifically, the PCa patients expressing high levels of PTEN but low levels of PI3Kδ (left panel). The PCa patients expressing low levels of PTEN but high levels of PI3Kδ (middle panel). The PCa patients expressing high levels of PTEN as well as high levels of PI3Kδ-S. FIG. 7B shows western blot analysis of PI3Kδ, PI3Kδ-S, PTEN, PAKT, AKT, pS6, S6 and β-actin levels in 22Rv1, LNCaP, MDA PCa 2b, HT29, A549 and MCF-7 cells transfected with nonsense/scrambled (NS) siRNA, siPTEN or pcDNA3-FLAG-PTEN plasmid (indicated as PTEN). FIG. 7C shows quantification of PI3Kδ, PI3Kδ-S, PTEN, pAKT, AKT, pS6 and S6 levels (of all experimental groups in FIG. 7B, normalized to NS controls) from 3-4 independent western blot results. The bar graphs were presented as mean±SD, and the significances (*p-value<0.05 in siPTEN vs. NS, and #p-value<0.05 in PTEN vs. NS) were calculated based on one-way ANOVA with Tukey's post hoc tests.

FIGS. 8A-8D show protein levels of PI3Kδ-L, PI3Kδ-S, and PTEN in endocrine and solid tumors. FIG. 8A shows western blots and FIG. 8B shows quantification of PI3Kδ-L, PI3Kδ-S and PTEN expression levels in PCa cell lines 22Rv1, PC-3, LNCaP, MDA PCa 2b, DU-145 and C4-2B. FIG. 8C shows Western blots and FIG. 8D shows quantification of PI3Kδ-L PI3Kδ-S and PTEN expression levels in colon (HT-29, SW620). lung (A549, H1299), and breast (MDA MB 231, MCF-7) cancer cell lines. GAPDH was used as an endogenous control for the western blots (representative images from n=3). The quantification data (in FIG. 8B and FIG. 8D were relative protein levels after normalization to GAPDH levels in corresponding cell lines.

FIGS. 9A-9C show SRPK1/2 inhibitor SRPIN340 reverses the aberrant splicing and sensitizes endocrine/solid tumor cells to PI3Kδ inhibitor Idelalisib. FIG. 9A shows RT-PCR assays for examining the PIK3CD-L and PIK3CD-S expression profiles in 22Rv1, LNCaP, MDA PCa 2b, HT29, A549 and MCF-7 cells in the presence of vehicle, 25 μM of Idelalisib, 25 μM of SRPIN340 and a combination of 25 μM Idelalisib and 25 μM SRPIN340. EIF1AX was used as an endogenous control for the RT-PCR assays. The PIK3CD-S/PIK3CD-L (S/L) ratios were determined as described in Methods. FIG. 9B shows western blot analysis of PAKT, AKT, pS6 and S6 protein levels in 22Rv1, LNCaP, MDA PCa 2b, HT29, A549 and MCF-7 cells. The western blot images of pAKT, AKT, pS6 and S6 were representative blot images from 3-4 independent repeats. The b-actin was used as an endogenous protein control. FIG. 9C shows quantification of pAKT, AKT, pS6 and S6 levels (of all experimental groups in FIG. 9B, normalized to NS controls) from 3-4 independent western blot results. The bar graphs were presented as mean±SD, and the significances (*p-value<0.05 in drug treatment vs. vehicle, *p-value<0.05 in Idelalisib/SRPIN340 vs. Idelalisib, and § p-value<0.05 in Idelalisib/SRPIN340 vs. SRPIN340) were calculated based on one-way ANOVA with Tukey's post hoc tests.

FIGS. 10A-10F show cell viability assays of and the endocrine/solid tumor cells under treatments of PI3Kδ inhibitor and/or SRPK1/2 inhibitor. Immunofluorescence assays were employed to visualize the expression levels of total PI3Kδ (green fluorescence) and PI3Kδ-S splice isoform (red fluorescence) in the tumor spheroids developed from FIG. 10A PCa cell lines (22rv1, PC3, LNCaP, MDA PCa 2b DU-145 and C4-2B), and endocrine/solid tumor cell lines (HT29 SW620, A549, H1299, MDA MB 231 and MCF-7). Nuclei were counterstained with DAPI (blue). FIG. 10B shows RT-PCR results revealing the expression profiles of PIK3CD-L and PIK3CD-S in all the tumor spheroids. FIG. 10C shows the bright-field images of tumor spheroids under treatment of vehicle, Idelalisib, SRPIN340 or Idelalisib/SRPIN340 combination for 5 days. FIG. 10D shows the average volumes of the endocrine/solid tumor spheroids after treatment of vehicle, Idelalisib, SRPIN340, or Idelalisib/SRPIN340 combination for 5 days. The volumes of spheroids were calculated based on the equation: V=4/3πR³, where V is volume and R is the radius averaged from 3-4 spheroids. The volume of the vehicle-treated spheroid in each cell line was defined as 100%. Therefore, the relative spheroid volume under treatment was determined by normalizing to its control (i.e. volume of drug-treated spheroid/volume of vehicle-treated spheroid×100%). Significances (**p-value<0.05 in drug treatment vs. vehicle, #p-value<0.05 in Idelalisib/SRPIN340 vs. Idelalisib, and ^{§} p-value<0.05 in Idelalisib/SRPIN340 vs. SRPIN340) were calculated based on one-way ANOVA with Tukey's post hoc tests. Data values represent mean±SD from 3-4 independent experiments. MTT assays for FIG. 10E 2D monolayer cultures and FIG. 10F shows 3D spheroid cultures of 22Rv1 LNCaP, MDA PCa 2b, HT-29, A549 and MCF-7 cells in the presence of vehicle, Idelalisib (25 μM), SRPIN340 (25 μM) and combination therapy (25 μM Idelalisib and 25 μM SRPIN340). Significances (*p-value<0.05 in drug treatment vs. vehicle, ^{#}p-value<0.05 in Idelalisib/SRPIN340 vs. Idelalisib, and ^{§} p-value<0.05 in Idelalisib/SRPIN340 vs. SRPIN340) were determined based on one-way ANOVA with Tukey's post-hoc tests. Data values represent mean±SEM from 5-6 independent experiments.

FIGS. 11A-11B show immunofluorescence assays of PI3Kδ and PI3Kδ-S in PCa and endocrine/solid tumors. Immunofluorescence images revealed the expression levels of PI3Kδ and PI3Kδ-S in 22 Rv1, PC-3, LNCaP, MDA PCa 2b, DU-145, C4-2B, HT-29, SW620, A549, HT-29, MDA MB 231, and MCF-7 spheroids.

FIG. 12 shows survival curves for endocrine/solid tumor patients expressing high and low levels of PI3Kδ. Significantly lower survival rates were observed in endocrine/solid tumor patients expressing high level PI3Kδ vs low level PI3Kδ. OS: overall survival; BCR: biochemical relapse; PFS: progression-free survival; RFS: relapse-free survival; DSS: disease-specific survival.

FIGS. 13A-13B show a combination of Idelalisib and SRPIN340 synergize the anti-tumor effects in the in vivo model.

FIG. 14 shows a high-throughput screening (HTS) for SMIs that inhibit PI3Kδ-S expressing PCa. Seven SMIs (AZD1080, Metformin HCl, PKI-587, BX517, TTP22, AZD8055, TIC10) were found to suppress PIK3CD-S expressing LNCaP, but not PIK3CD-L LNCaP. *P-value<0.05 based on ANOVA with Tukey's post hoc test.

FIG. 15 shows compounds differentially bound with PI3Kδ-S vs. PI3Kδ-L. Molecular docking simulation showing the interactions between seven compounds and PI3Kδ-L (brown) vs. PI3Kδ-S (green ribbons).

FIGS. 16A-16B show seven prostate cancer cell lines were grown and treated with vehicle and singe agents (Idelalisib, SPHINX31, MK-2206 2HCl, SB203580, Docetaxel, Enzalutamide, and Rapamycin) or combinations. The abbreviations for the drugs are listed as follows: IDE: Idelalisib, SPHINX: SPHINX31, MK: MK-2206 2HCl, DOC: Docetaxel, ENZ: Enzalutamide, RAP: Rapamycin). *, **, ***, ***: Significantly lower cell viability in drug treatment vs. vehicle control. ns: not significant.

An artisan of ordinary skill in the art need not view, within isolated figure(s), the near infinite distinct combinations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

PI3Kδ-L and PI3Kδ-S were Highly Expressed in PCa Specimens and Cell Lines.

To evaluate whether PI3Kδ and/or PI3Kδ-S splice variant can serve as a potential biomarker, a series of patient samples and cell lines derived from PCa, breast cancer, colorectal, lung and/or pancreatic cancers were subjected to IHC and RT-PCR assays for examining the expression profiles of PI3Kδ/PI3Kδ-S and PIK3CD-L/PIK3CD-S at protein and mRNA levels, respectively.

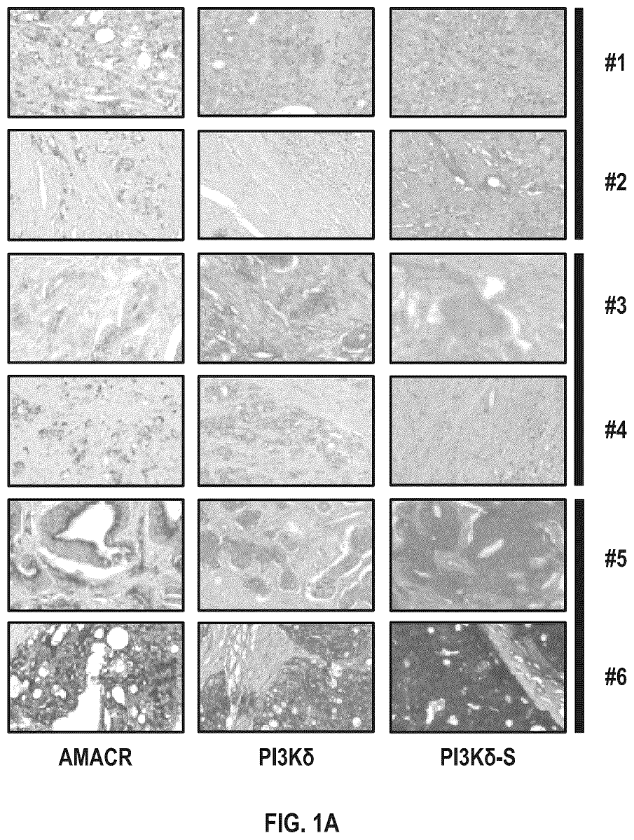

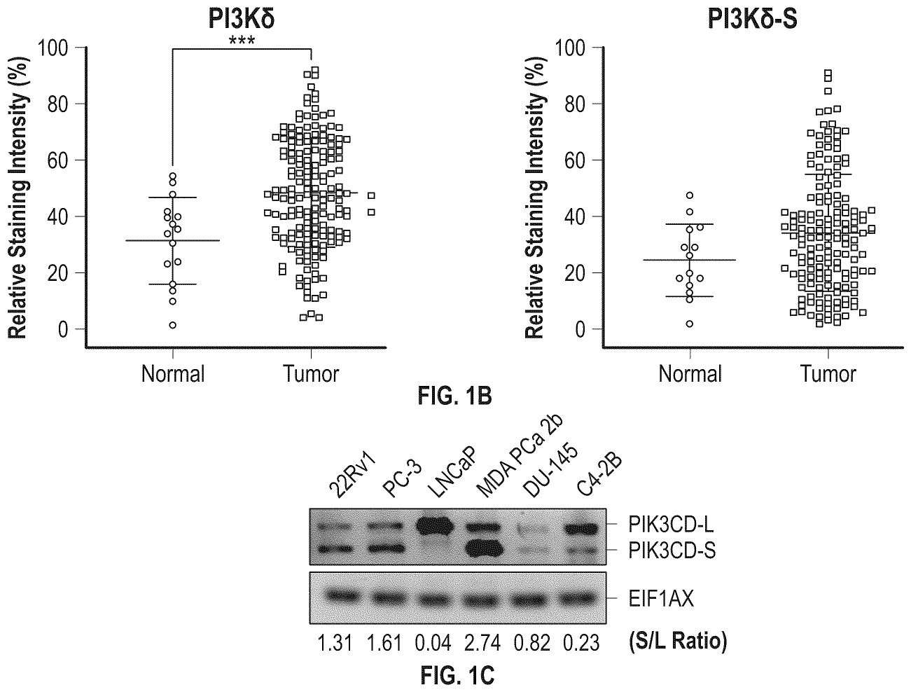

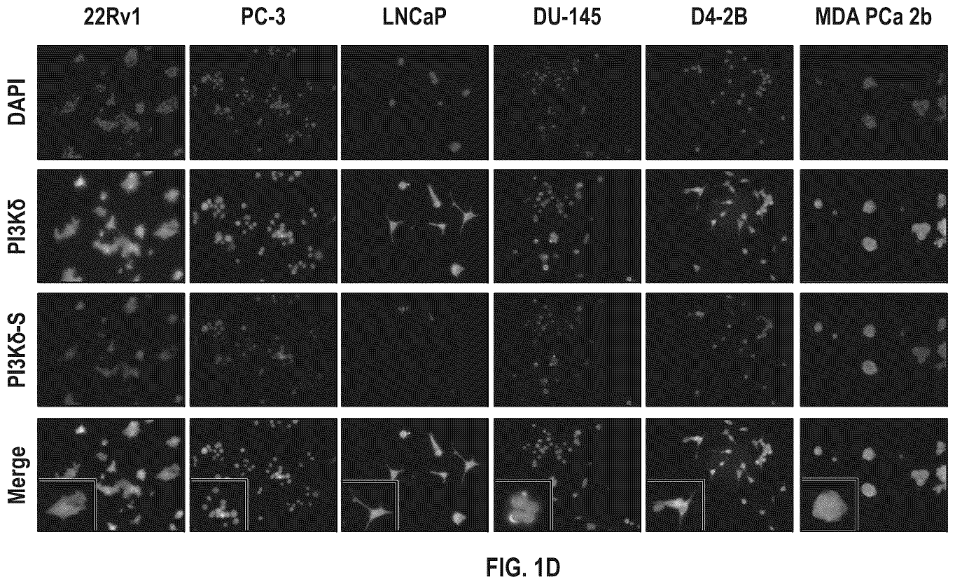

First, IHC assays were conducted on a TMA containing 160 cancerous and 16 adjacent normal tissues from 80 PCa patients, and 16 normal prostate tissues from 8 healthy individuals. The Gleason scores (GS) of the PCa samples on the TMA were ranging from 2+3 to 5+5. To assess the expression levels of PI3Kδ and it splice isoform in the PCa samples on the TMA, three independent IHC assays were performed to examine the expression profiles of α-methylacyl CoA racemase (AMACR, a potential PCa biomarker for its expression levels correlate with PCa progression), PI3Kδ and PI3Kδ-S splice isoform. As shown in FIG. 1A, three groups of PCa samples were revealed. Group 1 (patient #1 and #2 as examples) represented the PCa expressing AMACR, but not PI3Kδ nor PI3Kδ-S. Group 2 (patient #3 and #4 as example) represented the PCa expressing AMACR and PI3Kδ, but not PI3Kδ-S. Group 3 (patient #5 and #6 as examples) represented the PCa expressing AMACR, PI3Kδ, and PI3Kδ-S. Specifically, the IHC assay results revealed that almost 100% of PCa samples on the TMA expressed medium to high levels of AMACR. Among all the AMACR-positive PCa samples, 82.5% (132 out of 160) and 60% (97 out of 160) samples expressed moderate to high levels of PI3Kδ and PI3Kδ-S, respectively. Quantification of IHC images have further shown that PI3Kδ and PI3Kδ-S expression levels were higher in PCa specimens than the normal prostate tissues (FIG. 1B). These IHC results suggested that PI3Kδ and PI3Kδ-S may serve as potential biomarkers for PCa diagnosis.

A series of PCa cell lines with different pathological features were subjected to RNA purifications and RT-PCR assays to examine the expression profiles of PIK3CD-L (that encodes full-length PI3Kδ) and PIK3CD-S splice variant. PC-3 and DU-145 are androgen receptor (AR) negative PCa and represent androgen-independent PCa cell line models, LNCaP is an AR-positive and androgen-dependent PCa derived from lymph node. C4-2B (developed from LNCaP) and 22Rv1 are known as castration resistant prostate cancer (CRPC) cell lines, and MDA PCa 2b is an androgen-independent PCa cell line derived from bone metastasis of an African American (AA) patient. As shown in FIG. 1C, the RT-PCR results have revealed that the androgen-independent PCa cell lines MDA PCa 2b, PC-3, 22Rv1 and DU-145 have higher PIK3CD-S/PIK3CD-L (S/L) ratios, 2.74, 1.61, 1.31, and 0.82. The androgen-sensitive/dependent LNCaP predominately expressed full-length PIK3CD-L, with a very low S/L ratio of 0.04. In contrast, C4-2B, a CRPC cell line derived from LNCaP, expressed higher level of PIK3CD-S with a S/L ratio of 0.23 (˜6 time higher than the S/L ratio of parental cell line LNCaP). These results indicated that higher PIK3CD-S expression levels may correlate with more aggressive PCa phenotypes.

Next, immunofluorescence staining assays were conducted to visualize and verify the expression levels of PI3Kδ and PI3Kδ-S in the PCa cell lines described above. As shown in FIG. 1D, the six PCa cell lines demonstrated differential PI3Kδ (green fluorescence) and PI3Kδ-S (red fluorescence) expression profiles/levels. Specifically, nearly 100% of 22Rv1 expressed total PI3Kδ (green fluorescence) and PI3Kδ-S (red fluorescence). Detailly, the ratio of red to green fluorescence intensities was ˜3:5 (PI3Kδ-S: PI3Kδ-L+PI3Kδ-S), which is equal to PI3Kδ-S/PI3Kδ-L (S/L) ratio of 1.5 (calculated based on 3/2=1.5). PC-3 expressed red to green fluorescence ratio of ˜1:2, which is equal to a S/L ratio of ˜1. LNCaP expressed high level of PI3Kδ but very low level of PI3Kδ-S, with an average red to green ratio of ˜1:9 (equal to S/L ratio of ˜0.125). In DU-145 cells, the expression levels of PI3Kδ (green signals) was approximately two-fold higher than the PI3Kδ (red signals). Therefore, the average red to green ratios in DU-145 cells was ˜1:2 (which is equal to S/L ratio of ˜1). C4-2B cells expressed three-fold higher intensities of total PI3Kδ (green signals) than PI3Kδ-S (red signals), indicating that the red to green ratio was ˜1:3 that is equal to S/L ratio of 0.5. Whereas, MDA PCa 2b cells expressed high level of red fluorescence (PI3Kδ-S) signals, with ˜70% of fluorescence intensities when compared to the green signals (total PI3Kδ). The results suggested that MDA PCa 2b exhibits an average red to green ratio of ˜7.5:10, which is equal to S/L ratio of ˜3. Taken together, the immunofluorescence assays (FIG. 1D) showed similar trends of PIK3CD-S/PIK3CD-L (S/L) ratios as the results obtained from the RT-PCR assays (FIG. 1C) in these six PCa cell lines.

Both PI3Kδ-L and PI3Kδ-S were Expressed in Endocrine and Solid Tumor Specimens and Cell Lines.

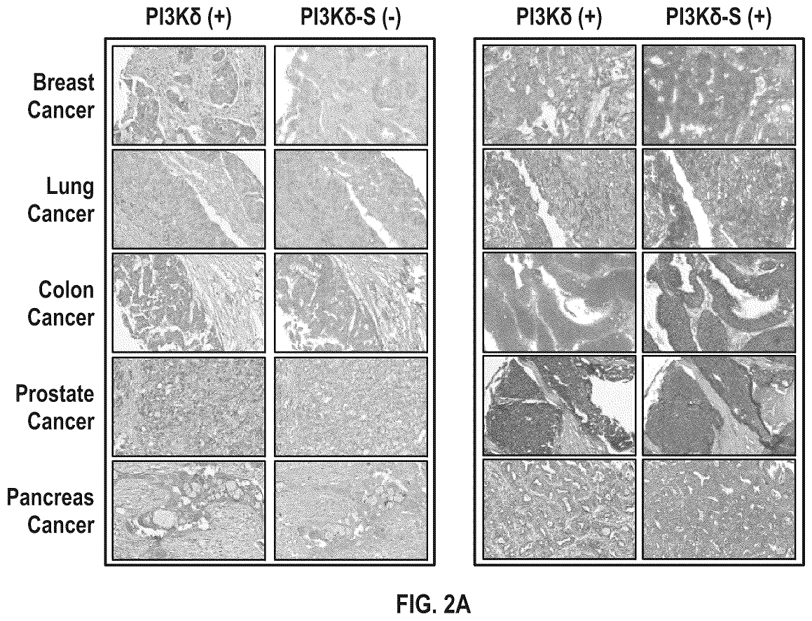

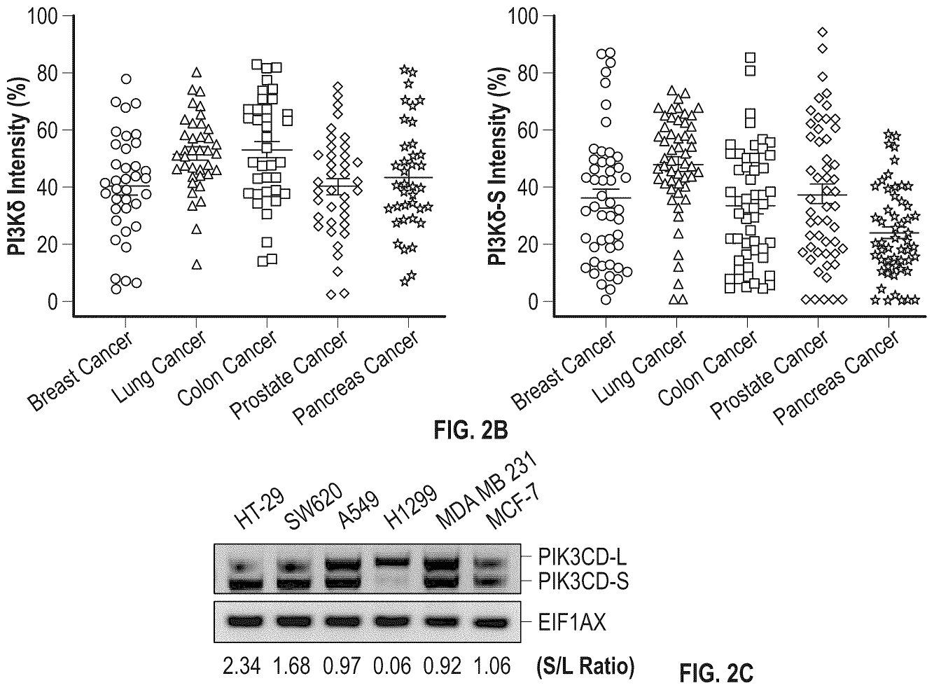

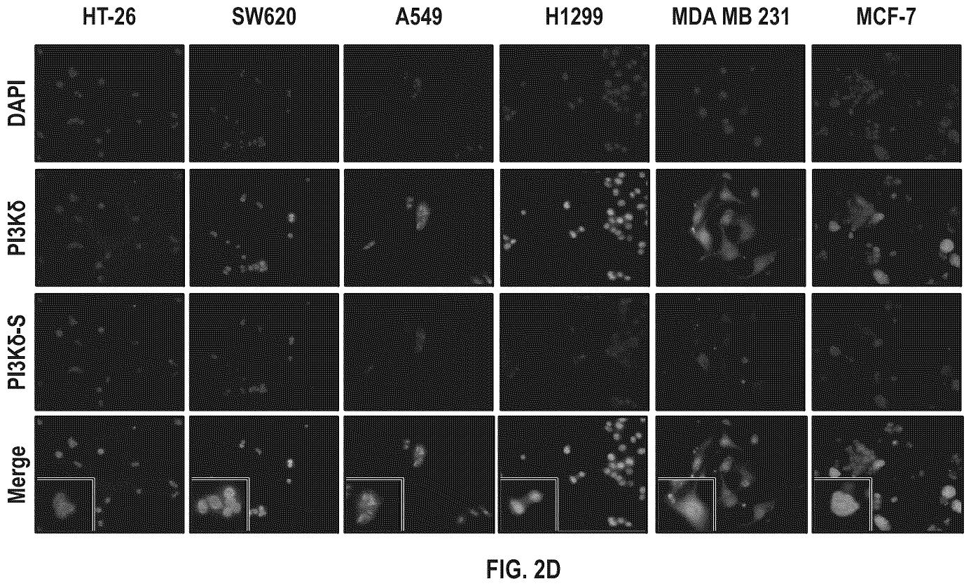

To further investigate whether PI3Kδ-L and PI3Kδ-S are generally expressed in solid tumors, IHC assays was performed on a TMAs containing cancerous specimens derived from endocrine cancer (breast, prostate, and pancreatic cancer), lung cancer and colorectal cancer patients. First, IHC results have revealed that PI3Kδ was expressed in majority of the breast, prostate, pancreatic, lung and colon cancer specimens on the TMA (FIG. 2A). Similar to the observation in FIG. 1, PI3Kδ-S levels were detected in subsets of the breast, lung, colon, prostate and pancreatic cancer specimens. Specifically, 20-30% of the PI3Kδ-positive cancer samples did not express PI3Kδ-S (FIG. 2A, left panel). Whereas, 70-80% of cancer specimens expressed both PI3Kδ (i.e. PI3Kδ+PI3Kδ-S) and PI3Kδ-S splice isoform (FIG. 2A, right panel). Quantification of IHC images further revealed that PI3Kδ was expressed at comparable levels (with average intensities ranging from 40% to 50%) in these cancers (FIG. 2B, left panel). IHC quantification also demonstrated that comparable expression levels of PI3Kδ-S were detected in breast, lung and colon cancers (with average intensities ranging from 35% to 45%), while a lower average PI3Kδ-S level (with average intensity of ˜20%) was detected in pancreatic cancer specimens (FIG. 2B, right panel).

To further evaluate PIK3CD-L and PIK3CD-S expression profiles in the in vitro endocrine/solid tumor cell models, the RNA samples purified from breast cancer cell lines (MDA MB 231 and MCF-7), colon cancer cell lines (HT-29 and SW620), and lung cancer cell lines (A549 and H1299) were subjected to RT-PCR assays. The RT-PCR results have shown differential PIK3CD-L and PIK3CD-S expression profiles between these cancer cell lines. Specifically, HT-29 and SW620 expressed higher levels of PIK3CD-S than PIK3CD-L (with S/L ratios of 2.34 and 1.68), A549, MDA MB 231 and MCF-7 expressed comparable levels of PIK3CD-L and PIK3CD-S (with S/L ratios of 0.97, 0.92 and 1.06, respectively), while H1299 predominately expressed PIK3CD-L (with S/L ratio of 0.06) (FIG. 2C).

Next, immunofluorescence assays were performed to visualize/verify the expression levels of PI3Kδ and PI3Kδ-S in the colon, lung and breast cancer cell lines described above. As shown in FIG. 2D, HT-29, SW620, A549, H1299, MDA MB 231 and MCF-7 expressed differential PI3Kδ (green fluorescence, PI3Kδ-L+PI3Kδ-S) and PI3Kδ-S (red fluorescence) expression profiles. Specifically, HT-29 exhibited a red to green fluorescence ratio of ˜1:1.3 (equal to S/L ratio of ˜3.3), SW620 exhibited a red to green fluorescence ratio of ˜0.75:1 (equal to S/L ratio of ˜3), A549 exhibited a red to green fluorescence ratio of ˜1:3.5 (equal to S/L ratio of ˜0.4), H1299 expressed a red to green fluorescence ratio of ˜1:10 (equal to S/L ratio of ˜0.11), MDA MB 231 demonstrated a red to green fluorescence ratio of ˜1:3 (equal to S/L ratio of ˜0.5), and MCF-7 expressed a red to green fluorescence ratio of ˜1:2.5 (equal to S/L ratio of 0.67). Consistent with the IHC and RT-PCR results, the immunofluorescence assays again confirmed that both PI3Kδ-L and PI3Kδ-S were expressed in HT-29 and SW620, A549, H1299, MDA MB 231 and MCF-7 with differential S/L ratios.

Correlation of PI3Kδ and PI3Kδ-S Expression Levels with the Cancer Aggressiveness.

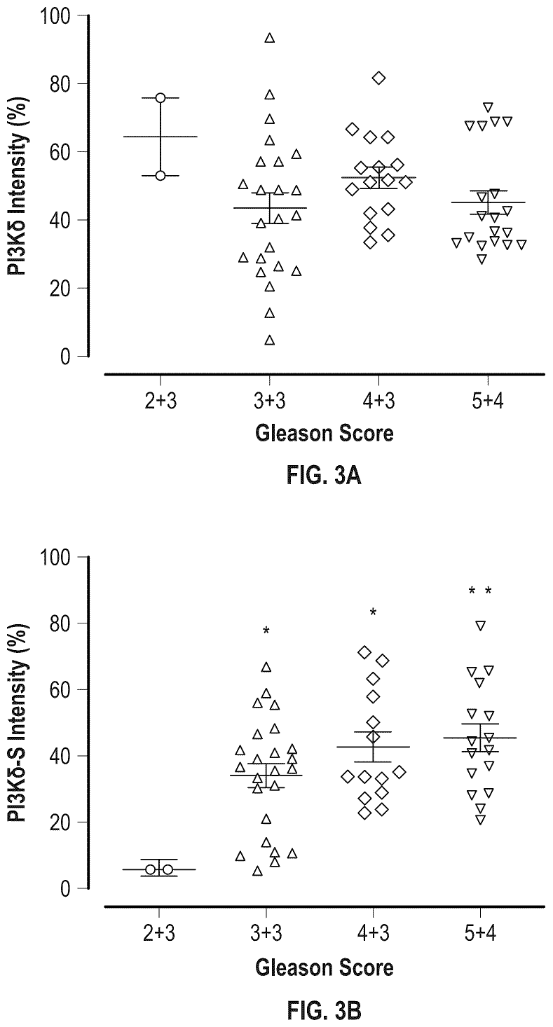

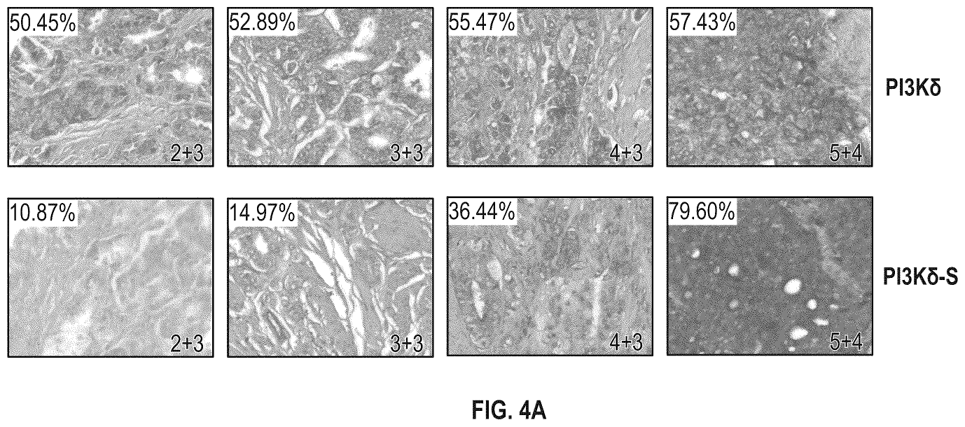

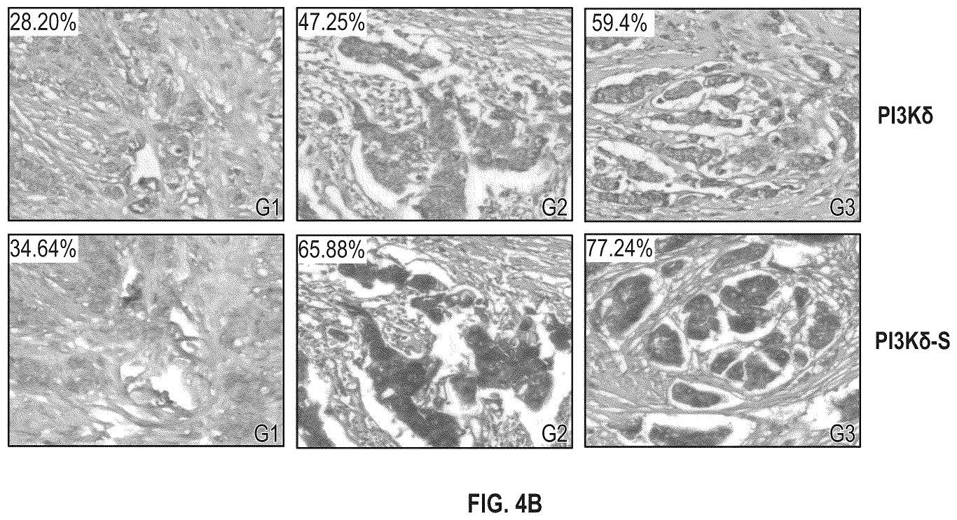

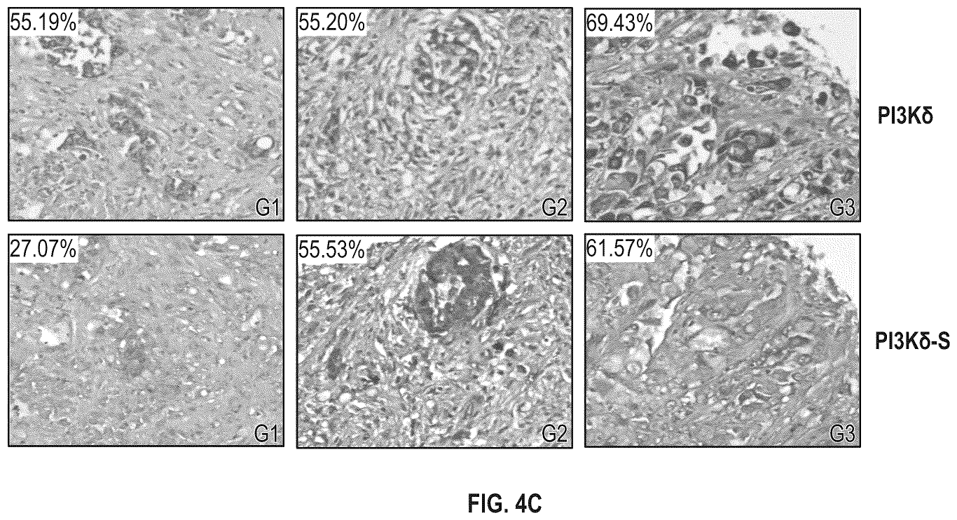

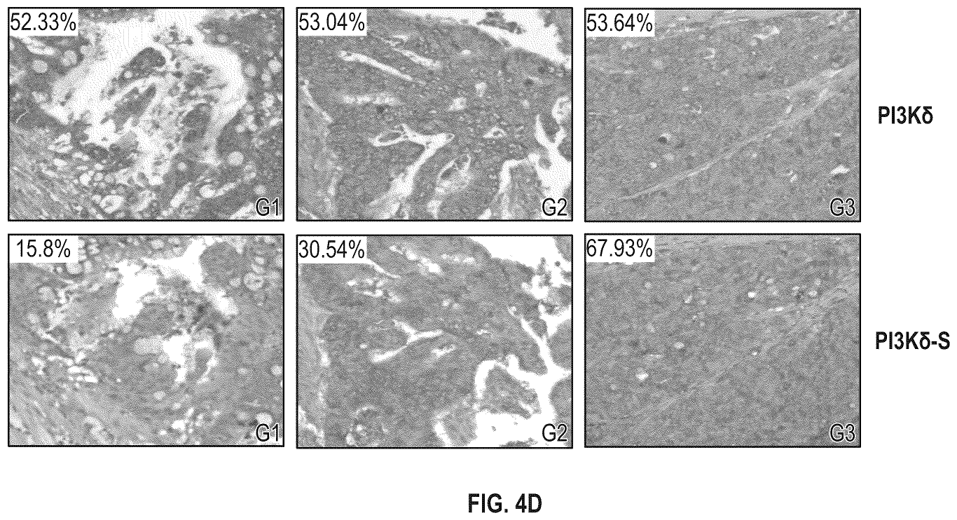

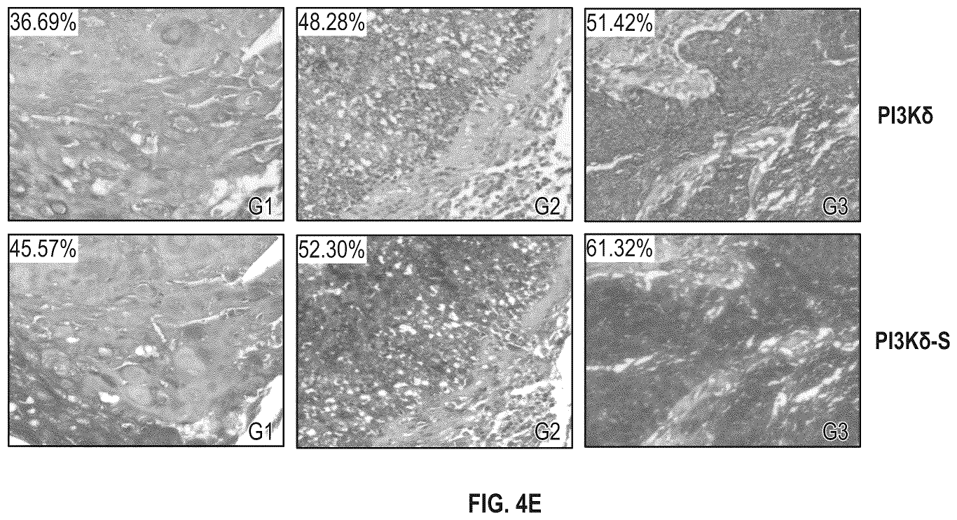

To evaluate the correlation of PI3Kδ/PI3Kδ-S with the tumor aggressiveness, IHC staining results of PI3Kδ and PI3Kδ-S in cancers were reviewed with different pathological features/states. Interestingly, comparable PI3Kδ expression levels were detected in PCa patient with Gleason Scores (GS) of 2+3, 3+3, 4+3, and 5+4 (FIG. 3A). In contrast, elevated PI3Kδ-S level seemed to be correlated with high-grade PCa. Specifically, the expression levels of PI3Kδ-S increased from GS 2+3, 3+3, 4+3 to 5+4 (FIG. 3B). Similarly, comparable PI3Kδ expression levels were observed in endocrine/solid tumor patients with different tumor grades (i.e. G1 vs. G2 vs. G3) (FIG. 4A-4E, top panels). However, higher expression levels of PI3Kδ-S splice isoform were observed in G3 vs. G2 and G2 vs. G1 in patients diagnosed with breast, pancreatic, colon and lung cancers (FIG. 4A-4E, bottom panels). Taken together, these results suggested that PI3Kδ may serve as a potential diagnostic biomarker for endocrine/solid tumors in general, while PI3Kδ-S splice isoform may particularly serve as a prognostic biomarker for cancer aggressiveness.

SiRNA Knockdown of PIK3CD-L and/or PIK3CD-S Inhibits ATK/mTOR Signaling in Endocrine/Solid Tumors.

Expression of PIK3CD-L or PIK3CD-S splice variant promotes oncogenic activation of AKT/mTOR signaling. To verify the functional effects of PIK3CD-L and PIK3CD-S in endocrine and solid tumors expressing PIK3CD-L and PIK3CD-S splice variant, the cancer cell lines were transfected with nonsense RNA (NS), siPIK3CD, siPIK3CD-L (siRNA targeting exon 20 of PIK3CD), or siPIK3CD-S (siRNA targeting junction of exon 19 and 21) for forty eight hours (48 h) then the transfected cells were harvested and subjected to western blot analysis for examining the protein levels of the AKT/mTOR signaling components.

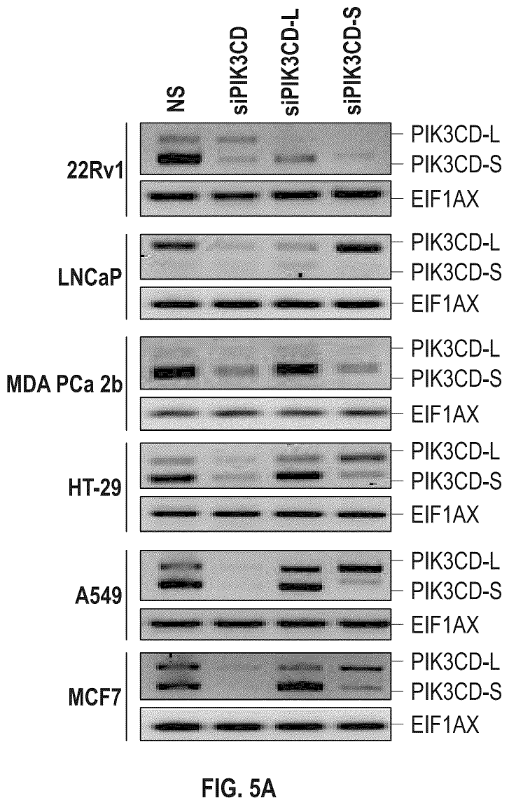

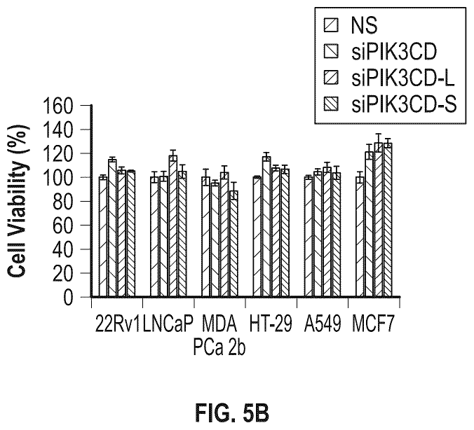

Androgen-sensitive PCa (LNCaP, with lowest PIK3CD-S/PIK3CD-L ratio), androgen-independent PCa (22Rv1 and MDA PCa 2b, with high PIK3CD-S/PIK3CD-L ratios), colon, lung and breast cancer cell lines (HT-29, A549 and MCF-7, with higher PIK3CD-S/PIK3CD-L ratios among each cancer types) were selected as in-vitro endocrine/solid tumor cell models for this experiment. First, RT-PCR assays confirmed the efficiencies and specificities of siRNA knockdown using siPIK3CD, siPIK3CD-L and siPIK3CD-S. Second, the cell viability assays performed upon siRNA knockdowns further confirmed that no toxicities were observed upon siRNA treatments (FIGS. 5A-5B).

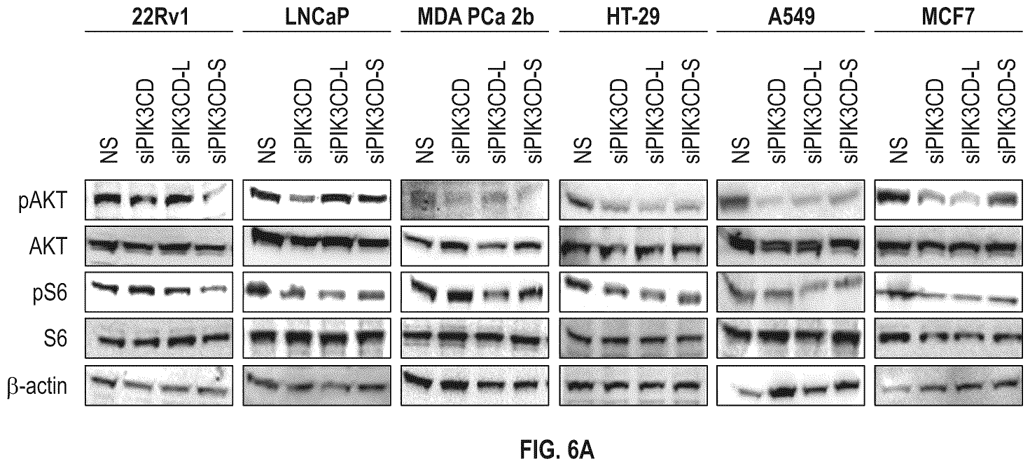

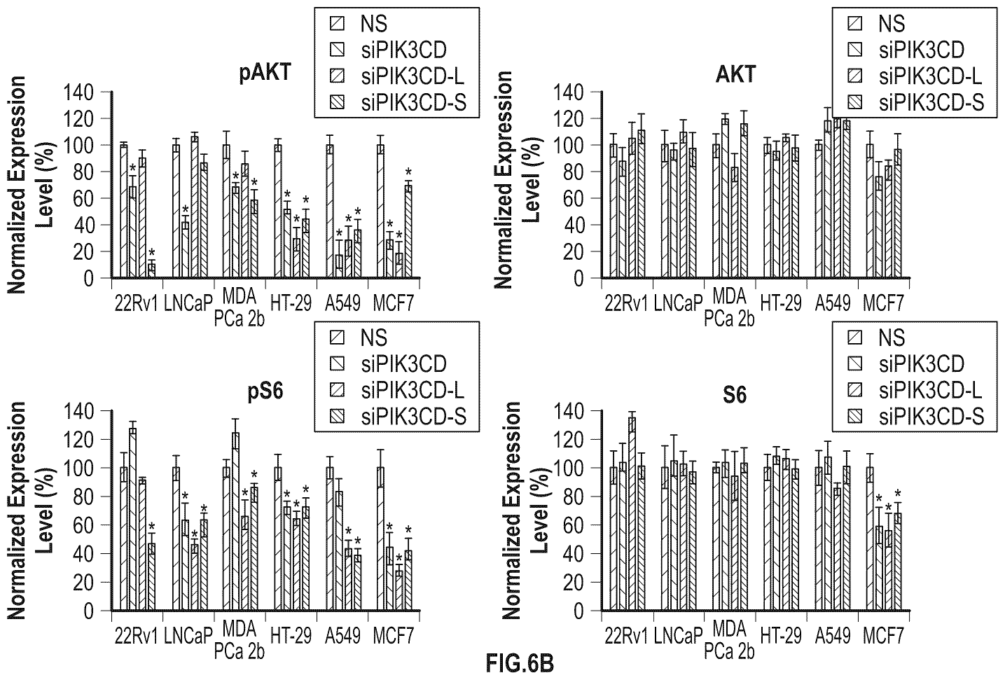

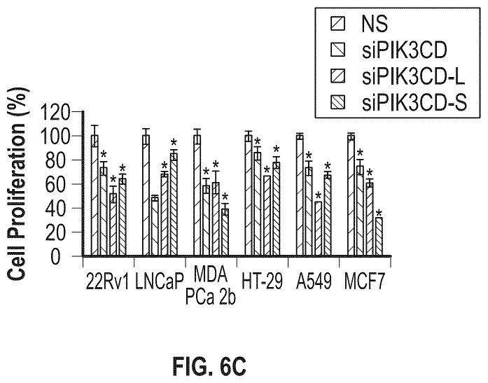

Upon the efficient/specific knockdown of total PIK3CD, PIK3CD-L or PIK3CD-S, the phosphorylation states of AKT are significantly reduced upon siRNA knockdown of total PIK3CD, PIK3CD-L or PIK3CD-S vs. NS, in all the endocrine/solid tumor cell lines. Additionally, a statistically significant reduction in the phosphorylation states of S6 were observed in siPIK3CD, siPIK3CD-L and siPIK3CD-S vs. NS transfected cancer cell lines FIGS. 6A-6B). Followed by siRNA knockdown of NS, total PIK3CD, PIK3CD-L or PIK3CD-S, all the cancer cells were harvested and then subjected to BrdU-based cell proliferation assays.

Specifically, PCa (22Rv1, LNCaP and MDA PCa 2b), breast (MCF7), colon (HT-29), and lung (A549) cancer cell lines were selected as in vitro endocrine/solid tumor cell models for this experiment. As seen in FIG. 6C, the phosphorylation states of AKT is significantly reduced upon siRNA knockdown of total PIK3CD, PIK3CD-L or PIK3CD-S vs. nonsense RNA (NS), in all the endocrine/solid tumor cell lines. Additionally, a moderate to significant reduction in the phosphorylation states of S6 were observed in siPIK3CD, siPIK3CD-L and siPIK3CD-S vs. NS transfected cancer cell lines. These results have suggested that: 1) both oncogenic PIK3CD-L and PIK3CD-S variants promote the activation of AKT/mTOR signaling pathway; and 2) molecular targeting PIK3CD-L and/or PIK3CD-S inhibits ATK/mTOR signaling, potentially serving as a novel therapeutic strategy for treating endocrine/solid tumors.

### Correlation Between PTEN and PI3Kδ Expression Levels in PCa Specimens.

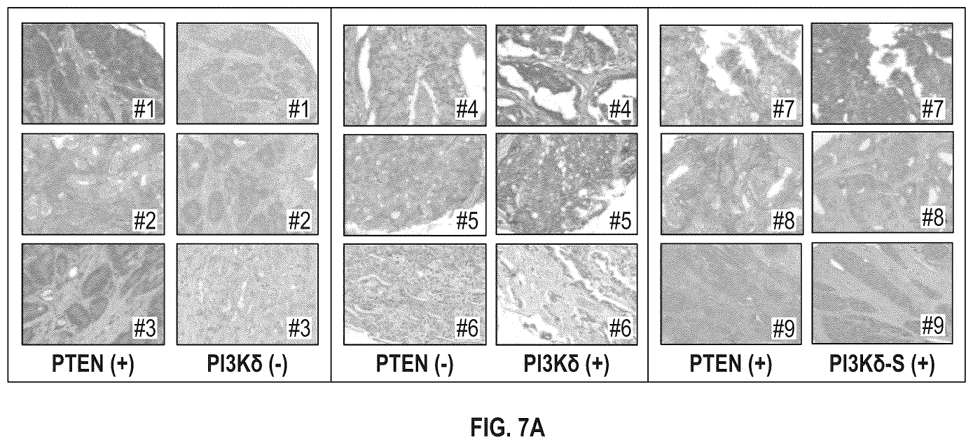

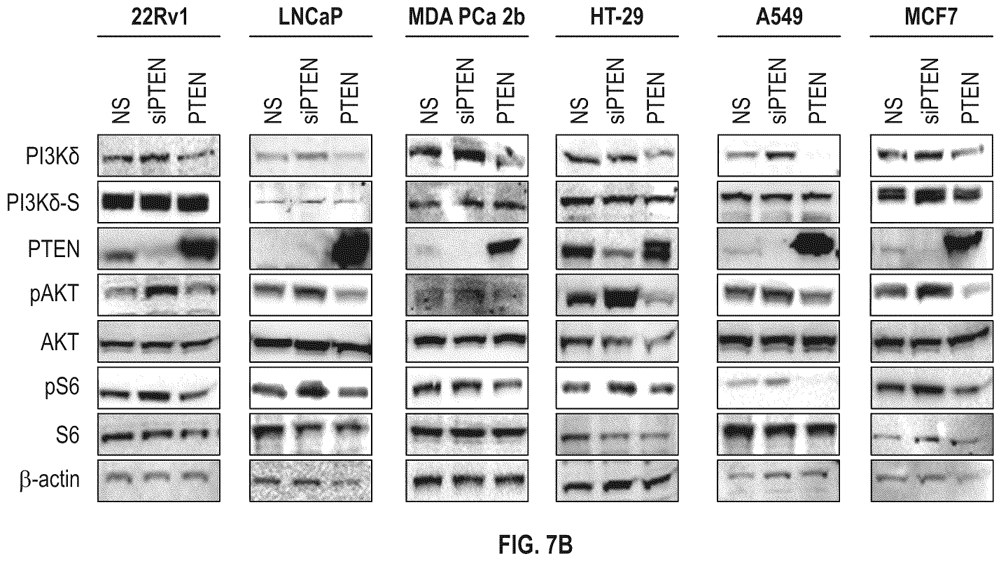

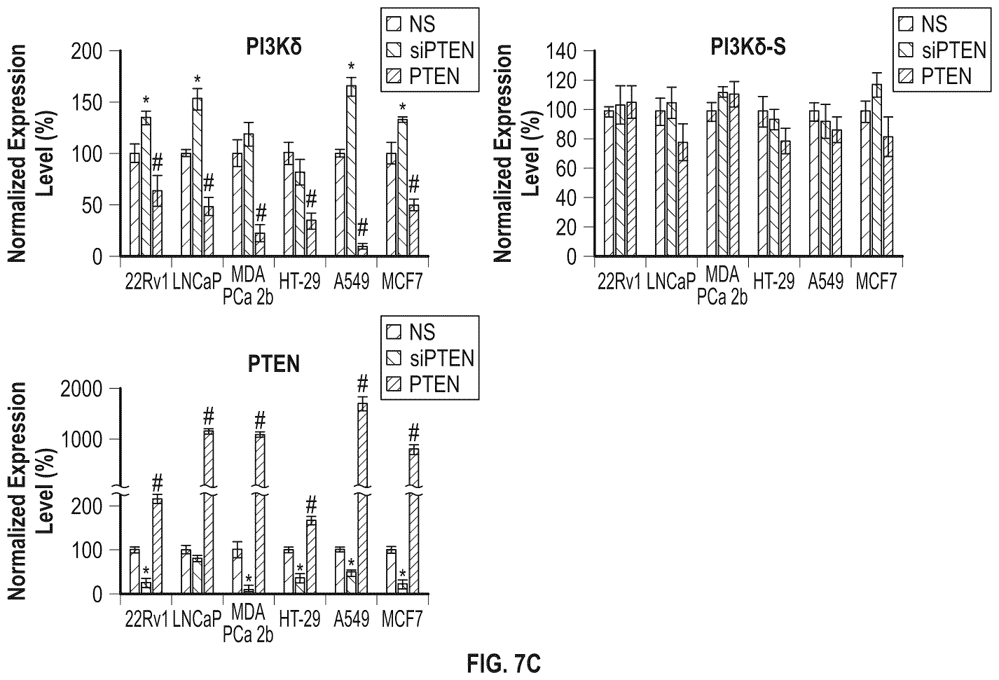

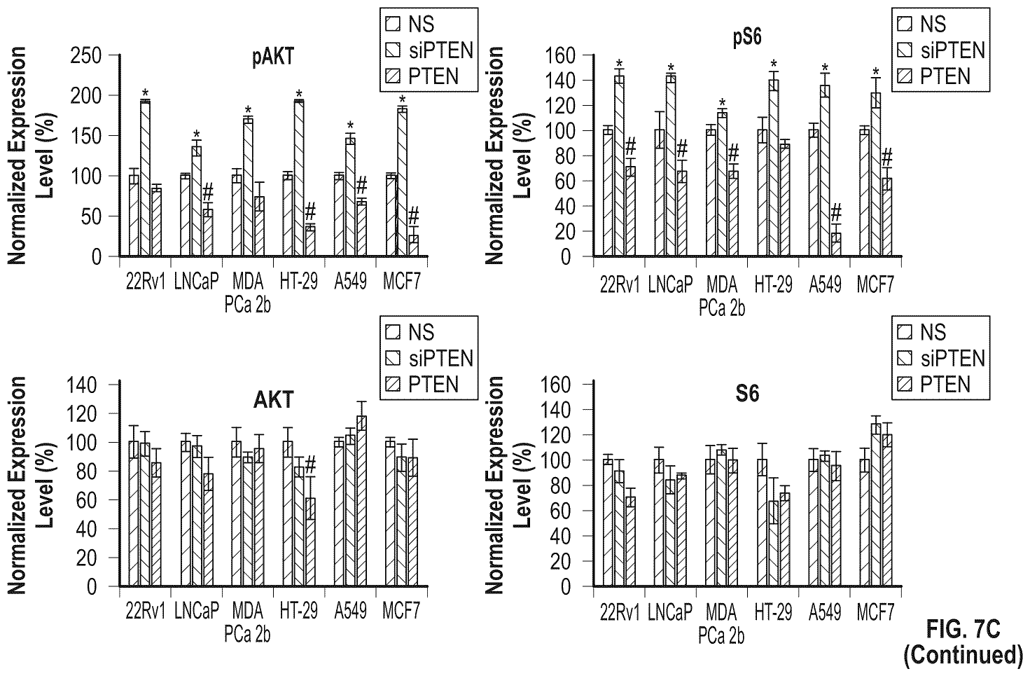

PTEN is a primary tumor suppressor gene, and it is frequently inhibited, deleted or loss-of-function in PCa. Previous studies have shown that overexpression of PTEN cause decease in PIK3CA expression, while knockdown of PTEN increase the PIK3CA expression. To verify whether there is a negative correlation/regulation between PTEN and PI3Kδ or PI3Kδ-S, IHC analysis of PTEN, PI3Kδ and PI3Kδ-S protein expression were performed using TMAs containing same cohort of the PCa specimens. Among the 160 PCa specimens, 111 PCa samples exhibited negative correlations between PTEN and PI3Kδ protein levels. Specifically, 98 PCa specimens expressed neglected/no PTEN but high levels of PI3Kδ. In contrast, 13 PCa specimens expressed PTEN but not PI3Kδ. The representative IHC images of PTEN and PI3Kδ with negative correlations were shown in FIG. 7A (left and middle panels). Surprisingly, IHC results have further revealed that 30 PCa specimens simultaneously expressed PTEN and PI3Kδ-S splice isoform (representative images in FIG. 7A, right panel), implicating that PI3Kδ-S expression may be not regulated/suppressed by PTEN in these PCa patients.

### Modulation of PTEN Expression Negatively Regulates Expression Levels and Activities of Full-Length PI3Kδ, but not PI3Kδ-S Splice Isoform.

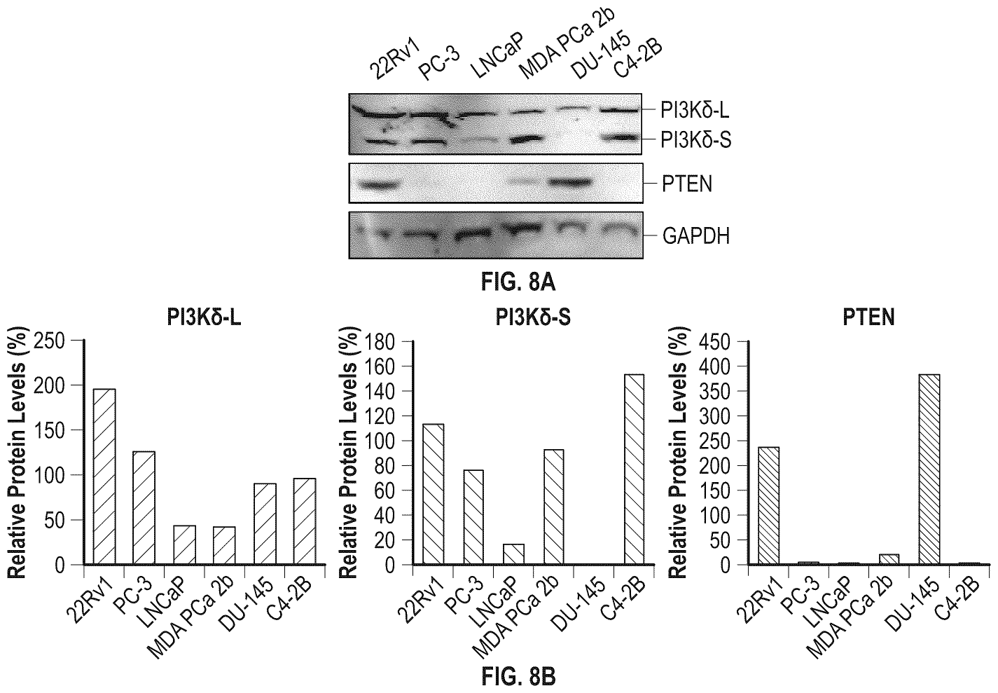

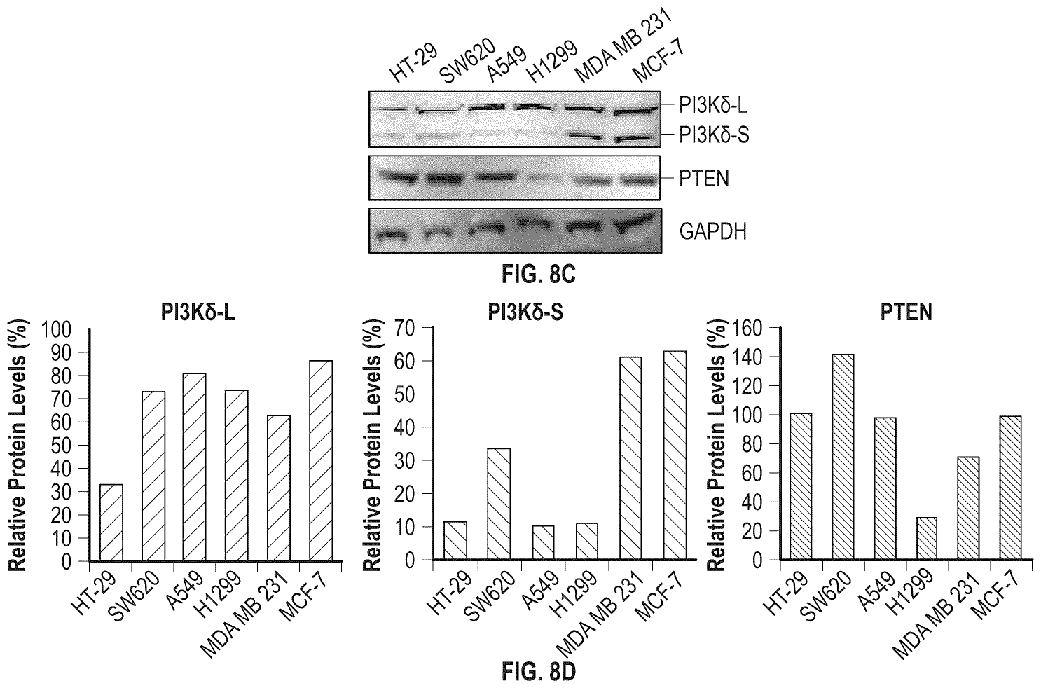

Except PC-3 and DU-145 (PTEN-negative PCa cell lines), other endocrine/solid tumor cells expressed low to high expression levels of PTEN (FIGS. 8A-7D). To further assess the functional roles of PTEN in regulating PI3Kδ and PI3Kδ-S expression, western blot analyses were performed in selected cancer cells transfected with NS, siPTEN, or pcDNA3-FLAG-PTEN plasmid (indicated as PTEN). As shown in FIG. 7B, the western blot results showed that siRNA knockdown of PTEN significantly increased the protein levels of PI3Kδ, while overexpression of PTEN drastically reduced PI3Kδ expression levels in 22Rv1, LNCaP, MDA PCa 2b, HT29, A549, and MCF-7 cells. Moreover, phosphorylation states of AKT and S6 were significantly enhanced upon siRNA knockdown of PTEN in cancer cells, suggesting AKT/mTOR signaling is activated upon loss of PTEN. In contrast, PTEN overexpression significantly inhibits phosphorylation of AKT and S6 in all the six cancer cell lines. These results confirmed that PTEN negatively regulates PI3Kδ expression and suppresses AKT/mTOR signaling. Interestingly, PI3Kδ-S expression levels remained comparable/unchanged between NS-transfected, siPTEN-transfected and PTEN-overexpressing cells. These results again suggested that PI3Kδ-S protein expression is independent from regulation/suppression by PTEN.

SRPIN340 Induces RNA Splice Switching and Inhibits AKT/mTOR Signaling in Combination with Idelalisib in Endocrine/Solid Tumor.

Idelalisib is an ATP-competitive inhibitor that specifically targets PI3Kδ and has potent anticancer effects against PI3Kδ-expressing cancer cells. Overexpression of PIK3CD-S splice variant in PCa confers AA PCa resistance to PI3Kδ inhibitor, such as Idelalisib. The synthesis of aberrant PIK3CD-S splice variant is likely mediated by the splicing factor SRSF2, and inhibition of SRSF2 by SRPK1/2 inhibitor SRPIN340 significantly sensitizes AA PCa to Idelalisib.

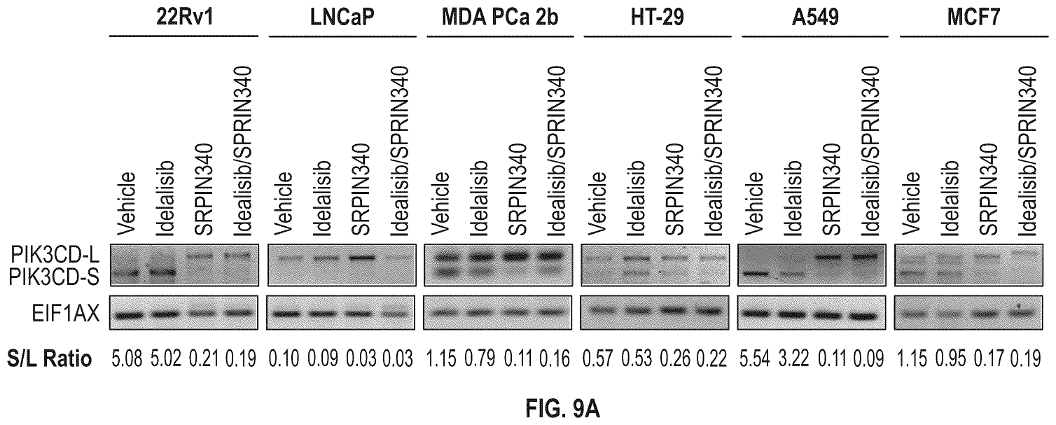

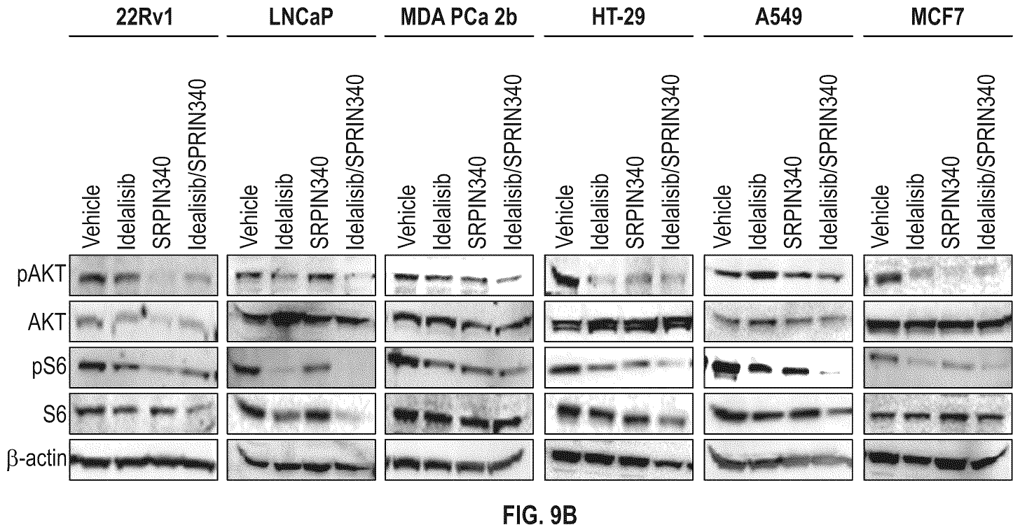

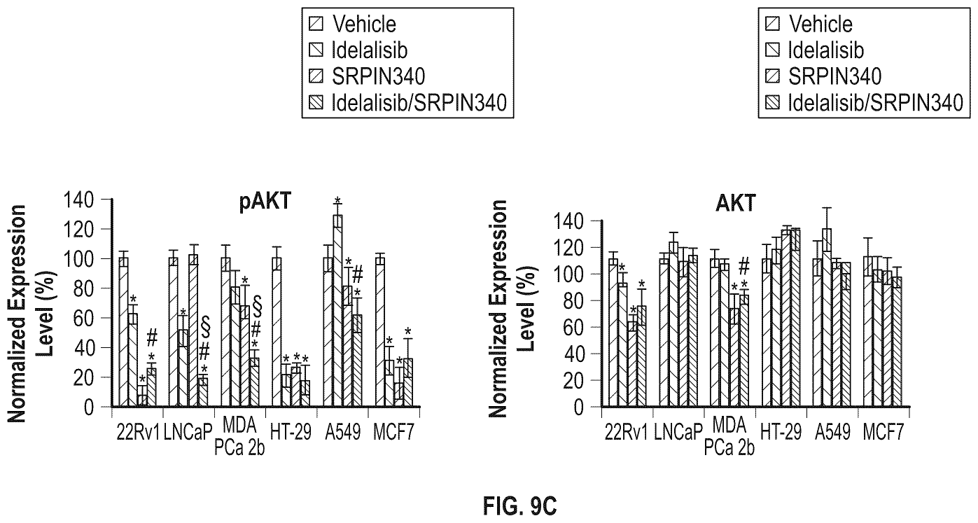

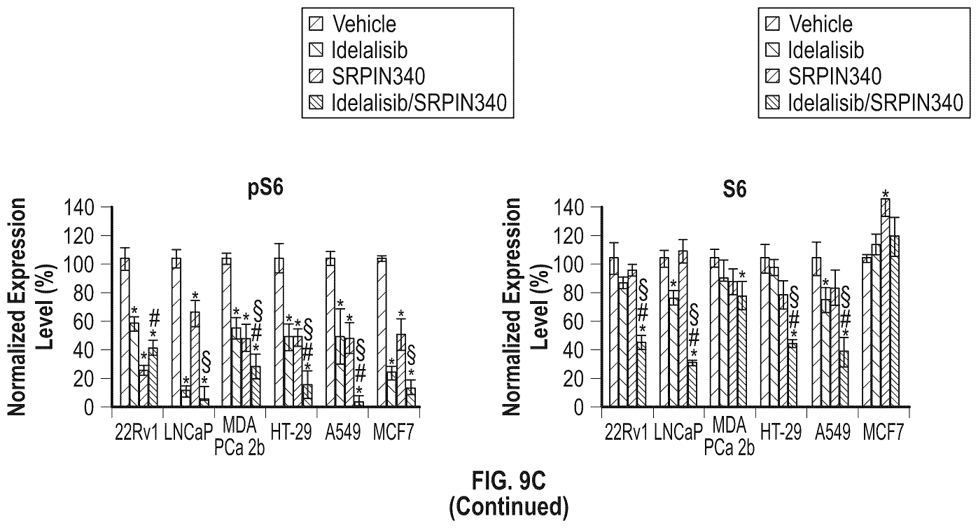

Except LNCaP, all the endocrine/solid tumor cell lines (22Rv1, MDA PCa 2b, HT29, A549, and MCF-7) expressed moderate to high levels of PI3Kδ-S (FIGS. 2C and 7A-7B). In theory, SRPIN340 treatment could inhibit the synthesis of PIK3CD-S variant through blocking the exon 20 skipping in PIK3CD pre-mRNA, thereby enriching the PIK3CD-L (which is sensitive to Idelalisib treatment) in these cancer cells. To validate this hypothesis, these cancer cell lines were grown and treated with vehicle, Idelalisib, SRPIN340, or a combination of Idelalisib and SRPIN340. After treatment for 48 hr, the cancer cells were harvested and subjected to RNA purification and RT-PCR assays. As anticipated, an ‘RNA splice switching’ pattern was observed in SRPIN340 vs vehicle treatments among the endocrine/solid tumor cell lines that express PIK3CD-S (22Rv1, MDA PCa 2b, HT-29, A549, and MCF-7). Compared to the vehicle control, SRPIN340 treatment inhibits the synthesis of PIK3CD-S, evident from the RT-PCR results (gel images in FIG. 9A) and the PIK3CD-S/PIK3CD-L (S/L) ratios were drastically decreased in all the cell lines (significant reduction of S/L ratios in SRPIN340 vs. vehicle, in FIG. 9A). In contrast, no changes in PIK3CD-S/PIK3CD-L (S/L) profiles/ratios were found in Idelalisib vs. vehicle treatments. These results strongly suggest that SRPIN340 inhibits PIK3CD-S synthesis and causes an ‘RNA splice switching’ to convert PIK3CD-S to PIK3CD-L, producing the full-length PI3Kδ that is sensitive to Idelalisib.

Next, the inhibitory effects of AKT/mTOR signaling pathway were examined in the presence of vehicle, Idelalisib, SRPIN340, or Idelalisib/SRPIN340 combination in the same cancer cell lines. As shown in FIG. 9B, pAKT and pS6 were significantly decreased in LNCaP (that predominately expressed PI3Kδ-L) in response to Idelalisib or Idelalisib/SRPIN340 combination compared to vehicle control. In contrast, no inhibition of PAKT and pS6 was observed in LNCaP cells under SRPIN340 treatment (reflecting the fact that negligible PIK3CD-S was present in LNCaP). For other endocrine/solid tumors (22Rv1, MDA PCa 2b, HT-29, A549, and MCF-7) that expressed both PI3Kδ-L and PI3Kδ-S, the phosphorylation states of AKT and S6 were significantly reduced in response to either Idelalisib or SRPIN340 treatment. And notably, the phosphorylation of AKT and pS6 were almost completely inhibited in cells treated with the Idelalisib/SRPIN340 combination (FIG. 9B).

### Combination of Idelalisib and SRPIN340 Effectively Inhibits Cancer Spheroids and Exhibits Potent Cytotoxicity in PIK3CD-S Expressing Cancers.

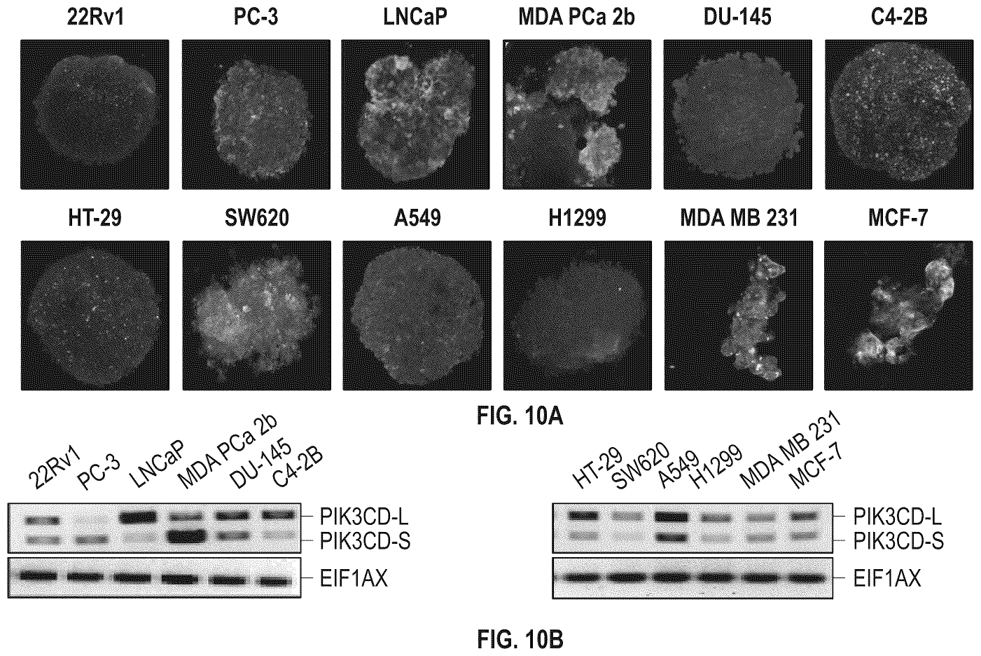

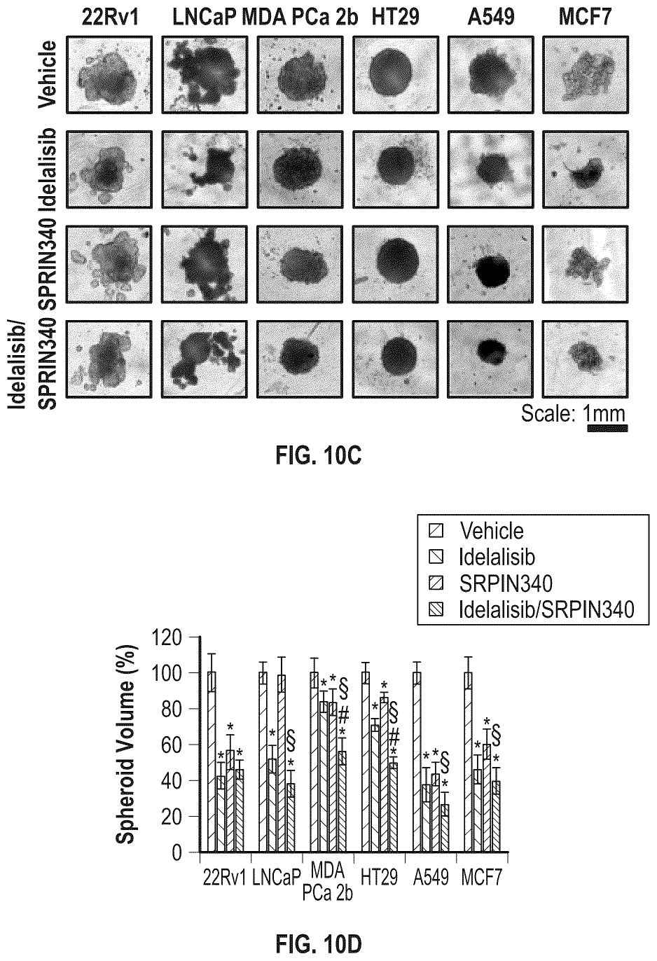

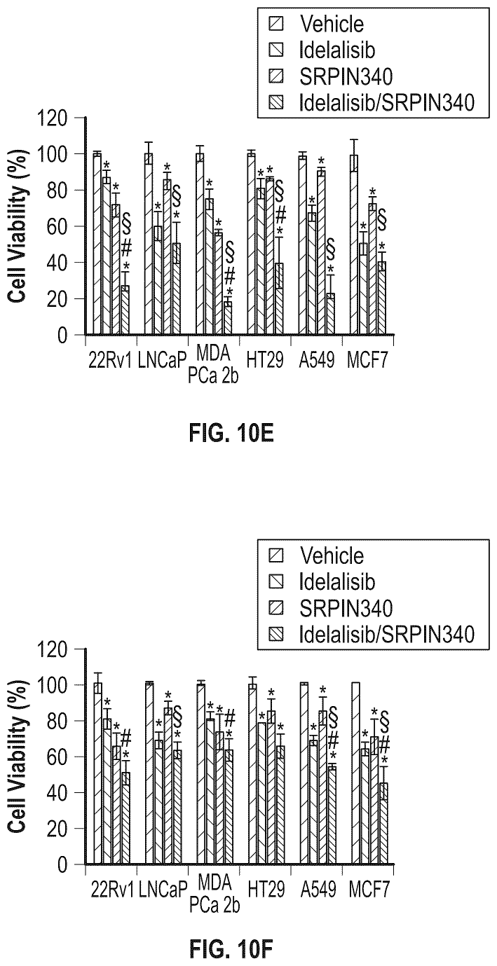

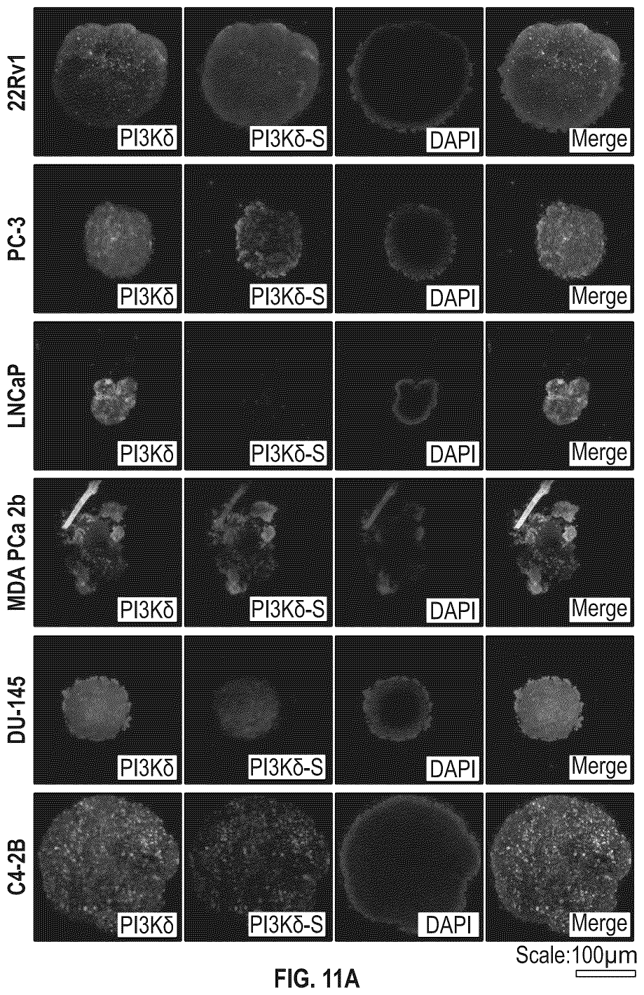

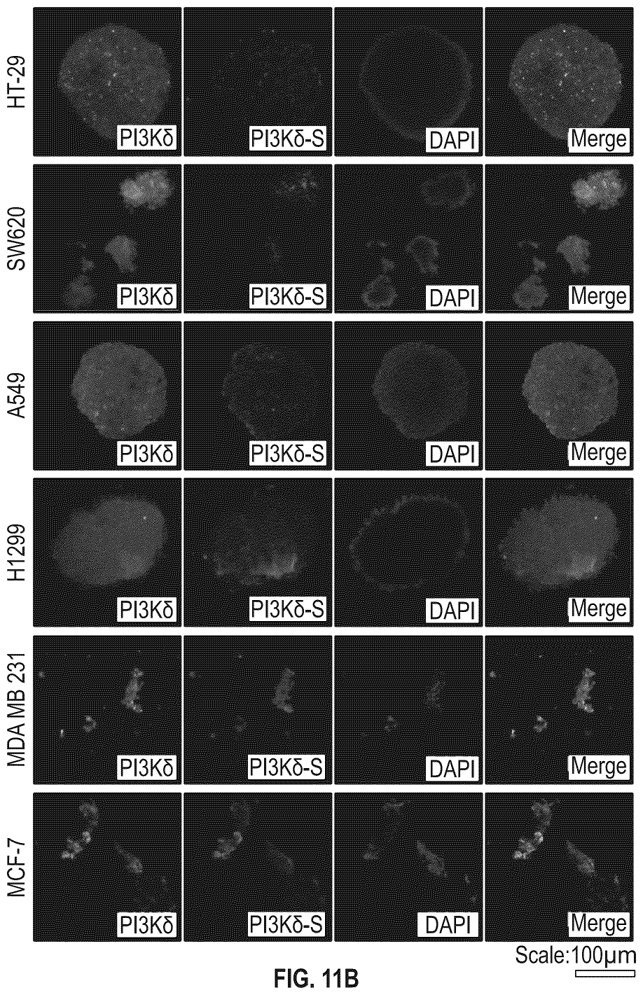

To evaluate the drug efficacies in the context of tumor microenvironment, 3D spheroid cultures in presence of vehicle, Idelalisib, SRPIN340, or Idelalisib/SRPIN340 combination were established. First, the 3D spheroid cultures developed from endocrine/solid tumor cell lines (22Rv1, LNCaP, MDA PCa 2b, HT-29, A549, and MCF-7) were assessed on day 5 using immunofluorescence assays to visualize the expression levels of PI3Kδ and PI3Kδ-S splice isoform. The green fluorescence represented the total PI3Kδ signals (total of PI3Kδ-L and PI3Kδ-S) and red fluorescence reflected the PI3Kδ-S signals, as shown in FIG. 10A and FIGS. 11A-11B. The immunofluorescence results from the 3D spheroids (FIG. 9A) have demonstrated similar expression levels/patterns of PI3Kδ-L and PI3Kδ-S as it was observed in regular 2D cell cultures (FIGS. 1D and 2D).

Second, all the spheroid cultures were harvested for RNA purifications and RT-PCR assays, to examine their expression profiles of PIK3CD-L and PIK3CD-S. The expression profiles of PIK3CD-L and PIK3CD-S in 22Rv1, PC-3, LNCaP, MDA PCa 2b, DU-145, C4-2B, A549, H1299, MDA MB 231, and MCF-7 spheroid cultures were similar to the expression patterns from the corresponding 2D cultures (FIG. 10A). However, HT-29 and SW620 spheroids expressed much lower PIK3CD-S levels than their 2D monolayer cultures (FIG. 10B vs. FIG. 2C).

Next, the efficacies of PI3Kδ inhibitor and SRPK1/2 inhibitor were examined (as single agents or in combination) on inhibiting the 2D monolayer and 3D spheroids in vitro. Specifically, the tumor spheroids were cultured in the presence of vehicle, 25 μM of Idelalisib, 25 μM of SPRIN340, or a combination of Idelalisib and SRPIN340. After drug treatments for 5 days, the tumor spheroids exhibited differential responses upon different treatments. In general, all the tumor spheroids responded to Idelalisib with significant reduction (i.e. 30-60% reduction) in tumor spheroid sizes/volumes, except MDA PCa 2b (FIGS. 10C and 10D). After SRPIN340 treatment for 5 days, all the tumor spheroids exhibited decreased spheroid sizes/volumes (i.e. 20-60% reduction), except LNCaP (FIGS. 10C and 10D). The differential drug responses reflect the fact that LNCaP almost exclusively expressed PI3Kδ-L (not responding to SRPIN340) while MDA PCa 2b predominantly expressed PI3Kδ-S (that is resistant to Idelalisib). However, significant/enhanced reduction in tumor spheroid sizes/volumes of all cancer lines were observed in the presence of Idelalisib/SRPIN340 combination, suggesting a synergistic drug effect of combining Idelalisib with SRPIN340 on inhibiting the PI3Kδ-L/-S expressing tumor spheroids (FIGS. 10C and 10D).

MTT assays were further performed to examine the drug effects on the cell viabilities of the regular 2D cultures and 3D spheroid cultures. Specifically, 22Rv1, LNCaP, MDA PCa 2b, HT-29, A549, and MCF-7 cells were treated with vehicle, Idelalisib, SPRIN340, or Idelalisib/SRPIN340 combination for 48 h (for 2D cultures) or 5 days (for 3D spheroid cultures) then subjected to MTT assays. As shown in FIG. 10E, the cell viabilities were significantly reduced in response to 25 μM of Idelalisib or 25 μM of SPRIN340 in all cell lines, except LNCaP in the presence of SPRIN340. Notably, a significant synergistic drug effect (with 50-80% decreases in cell viabilities) was observed in all cancer cell lines when treated with Idelalisib/SRPIN340 combination (FIG. 10E). Similar to the MTT assay results in the 2D cell cultures, Idelalisib or SRPIN340 has exerted moderate inhibitory capacities (with 15-35% decrease in cell viabilities) in all spheroid cultures. However, synergistic drug effects (with 40-55% reduction in cell viabilities) were observed in all the tumor spheroids treated with Idelelisib/SRPIN340 combination (FIG. 10F).

Emerging evidence has revealed that PI3Kδ is expressed not only in hematologic cancers, but also highly expressed in solid tumors. Particularly, previous studies have shown that PI3Kδ is overexpressed in PCa cell lines (DU-145, 22Rv1, PC-3, LNCaP and MDA PCa 2b), breast cancer lines (MDA MB 231 and MCF-7), colon cancer lines (SW620 and SW480) and lung cancer cell lines (A549, H1975, PC9 and H1650). Consistent with the previous studies, the data again confirmed that PI3Kδ is expressed in LNCaP, DU-145, PC-3, 22Rv1, MDA PCa 2b, MDA MB 231, MCF-7, SW620 and A549. Additionally, C4-2B (a castration resistant prostate cancer cell line) and H1299 (lung cancer cell line) have also shown with high levels of PI3Kδ. Notably all these endocrine/solid tumor cell lines also expressed PI3Kδ-S splice isoform (based on the IHC, immunofluorescence, and RT-qPCR analyses). To date, this was the first attempt to investigate the expression profiles of full-length PI3Kδ-L and PI3Kδ-S splice isoform in endocrine/solid tumor patient samples and cell lines. Given the fact that PI3Kδ and PI3Kδ-S exhibit differential oncogenic activities, the data suggested that a potential of utilizing the PIK3CD-S/PIK3CD-L (or PI3Kδ-S/PI3Kδ-L) expression profile as an index to evaluate the tumor aggressiveness in the endocrine cancers.

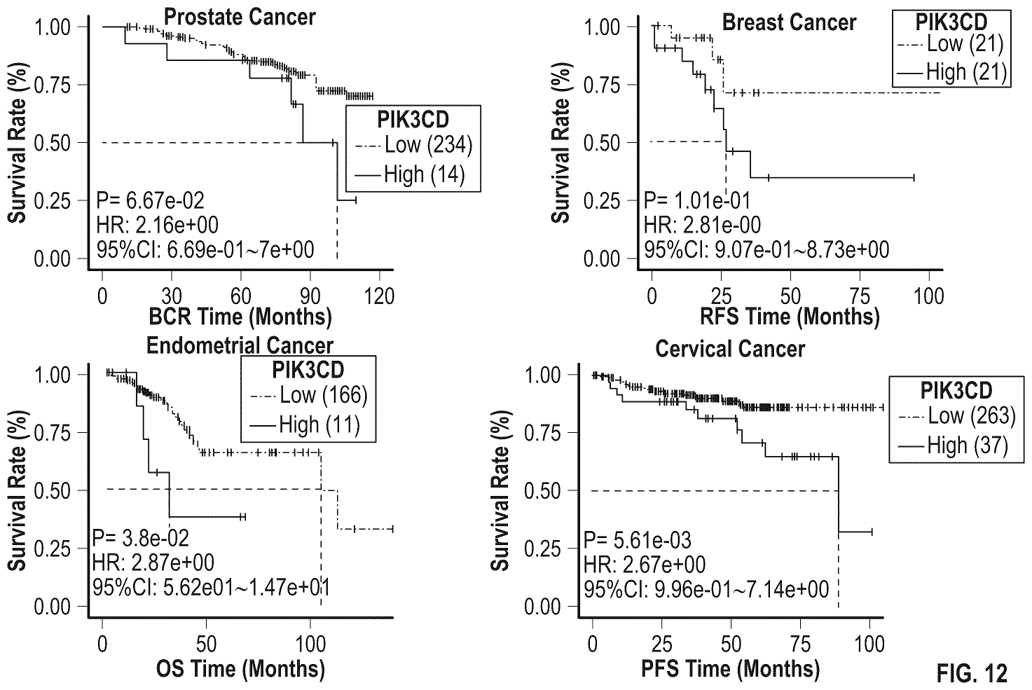

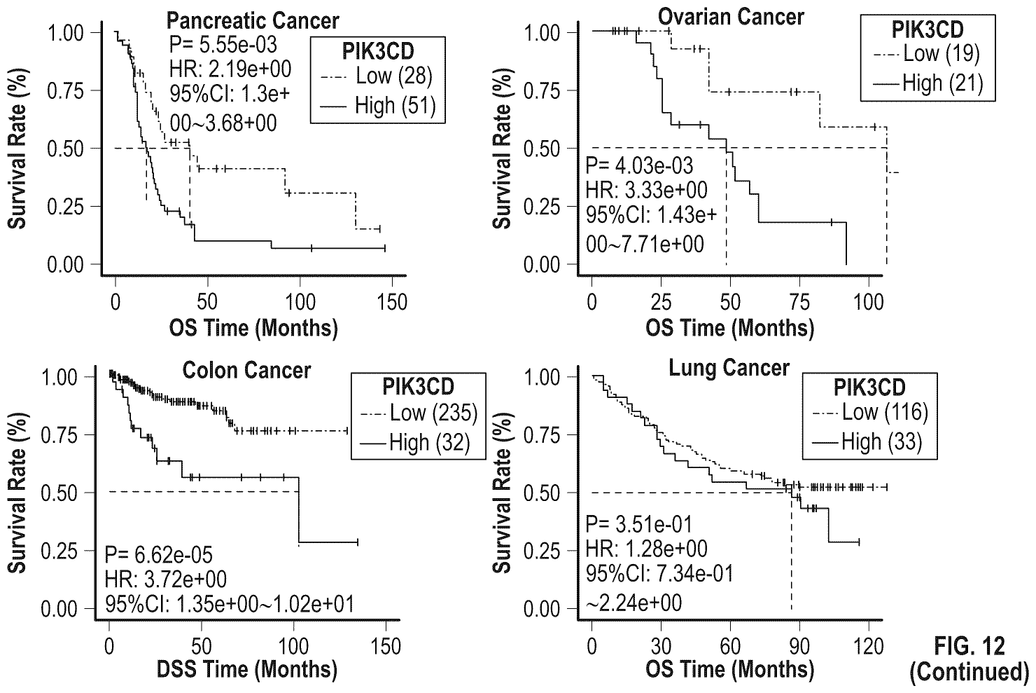

To further evaluate the potential of PI3Kδ-L and/or PI3Kδ-S as a diagnostic/prognostic biomarker, a survival analysis was performed. By employing the PanCanSurvPlot program (https://smuonco.shinyapps.io/PanCanSurvPlot/), possible correlations between PI3Kδ expression levels and cancer patient survival rates have been revealed. Specifically, higher expression levels of PI3Kδ (based on the RNA-seq data from TCGA database) appears to be correlated with poorer survival rates in selected patient cohorts with endocrine cancers (prostate, breast, pancreatic, ovarian, endometrial and cervical) or other solid tumors (colon and lung cancer) (FIG. 12). PIK3CD-S is a more oncogenic splice variant (compared to PIK3CD-L), and expression of PI3Kδ-S confers a drug resistance phenotype in PCa. Further bioinformatic efforts (i.e. retrieving RNAseq data to precisely define PIK3CD-L and PIK3CD-S expression levels in patients) may facilitate understanding on whether PIK3CD-S/PIK3CD-L or PI3Kδ-S/PI3Kδ-L expression ratios would correlate with the poorer survival rates and/or disease aggressiveness (i.e. drug resistance, recurrence, metastasis, and etc.) in endocrine cancers.

The tumor suppressor PTEN plays a critical role in regulating PI3K/AKT/mTOR signaling. Mutations and/or loss-off-function in PTEN are frequently found in various cancers, including endocrine/solid tumors, such as PCa, breast, colon, and lung cancers. A negative regulation between PTEN and PI3K has been highlighted in several cancers. In PCa, it has been shown that PTEN suppresses the expression of ARID4B, repressing the transcriptional activation of PIK3CA and subsequently inhibiting the PI3K/AKT signaling. In human nasopharyngeal carcinoma cells, siRNA knockdown of PTEN resulted in upregulation of PI3K (at mRNA and protein levels) and activation of PI3K/AKT signaling, while suppressing tumor suppressor FOXO3a. On the other hand, previous study further showed that PI3K levels may also modulate the activities of PTEN. Specifically, siRNA knockdown of PIK3CD activated PTEN activity. Whereas, ectopic expression of PI3Kδ resulted in suppression of PTEN activity, consequently suppressing AKT signaling and inhibiting cell proliferation in PCa and breast cancer cells. Similar to the previous findings, the results have also confirmed that inhibition of PTEN by siRNA caused upregulation of PI3Kδ expression and activation of AKT/mTOR signaling, evident from the increased pAKT and pS6 levels. Conversely, ectopic expression of PTEN resulted in suppression of AKT/mTOR signaling (i.e. reduced phosphorylation states of AKT and S6) (FIG. 7B). Additionally, the data have further revealed that overexpression of PTEN also reduced PI3Kδ protein expression level.

To date, the mechanisms underlying the PTEN-independent PI3Kd-S expression remain unknown. One of the possible mechanisms is: PI3Kδ-S expression levels are determined by the synthesis of PIK3CD-S, an aberrant splice variant resulted from a SRSF2-mediated exon 20 skipping event in PIK3CD pre-mRNA. This aberrant RNA splicing process is independent from regulation by PTEN, and therefore, the PI3Kδ-S levels could solely depend on the activities of SRSF2 in each cell lines. Further investigation of the upstream regulators of PTEN (i.e., p53, EGFR1, PPAR-g, SPRY2, and etc.) in each cell lines may help to elucidate the molecular mechanisms underlying the PTEN-independent PI3Kδ-S protein expression.

Furthermore, siRNA knockdowns of total PIK3CD variants, PIK3CD-L and PIK3CD-S showed differential inhibitory effects on AKT/mTOR signaling, possibly due to the differential S/L ratios in different cancer cell lines. Overall, siPIK3CD knockdown demonstrated good inhibition in AKT/mTOR signaling in general (FIG. 6A-6C). Notably, siRNA knockdown of PIK3CD-S has shown superior inhibition of AKT/mTOR signaling (i.e. significant reduction of pAKT and pS6 levels) in 22Rv1 and MDA PCa 2b, reflecting the higher sensitivities upon siPIK3CD-S due to their higher S/L ratios (1.31 and 2.74, respectively).

Accumulating evidence has suggested that aberrant mRNA splicing may represent one of the genetic mechanisms mediating drug resistance in cancers. The present disclosure provides a novel therapy by combining PI3Kδ inhibitor with splicing inhibitor. As shown in FIGS. 10C-10F, this drug combination generated a significantly synergistic effect on inhibiting the tumor growths and viabilities in the 2D monolayer and 3D spheroid cultures derived from the endocrine/solid tumor cell lines. To date, this is also the first report to systemically apply SRPK1/2 inhibitor (SRPIN340) for sensitizing the drug resistant endocrine/solid tumors. This drug combination may represent a novel therapy for treating Idelalisib-resistant cancers, with non-hematologic or hematologic origin. Future efforts of employing oligonucleotide therapy (such as antisense oligonucleotides, ASO and splice switching oligonucleotides, SSO), targeting critical splicing modulator (such as SF3B1, a core component in spliceosome), or screening/developing compounds specifically inhibiting PI3Kδ-S activity, may further warrant the development of novel therapies for overcoming the Idelalisib resistance in the PI3Kδ-expressing endocrine cancers.

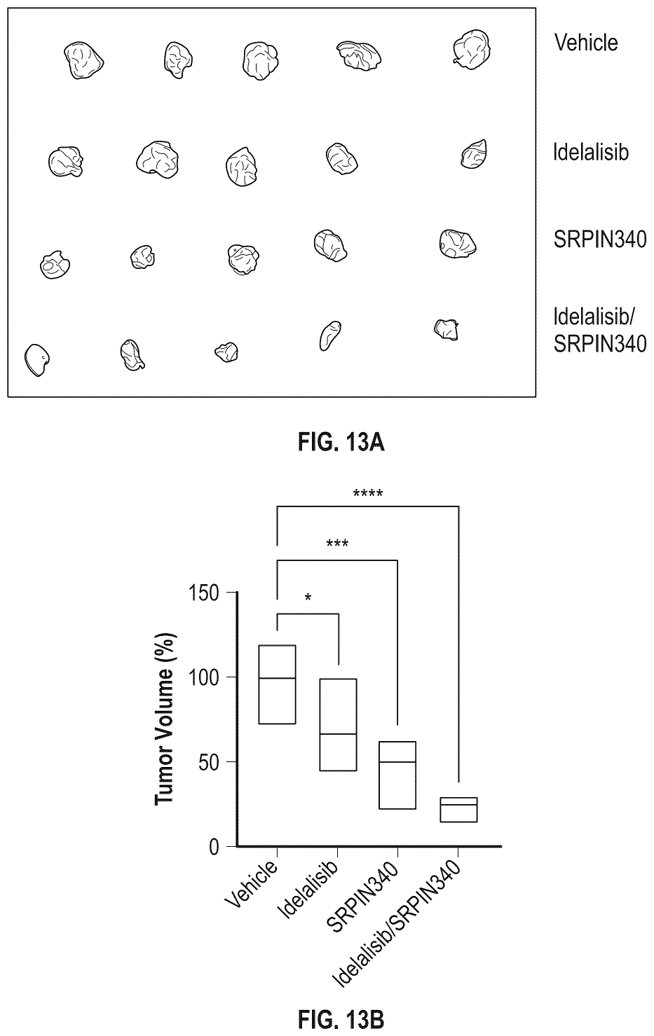

FIGS. 13A-13B evidence that a combination of Idelalisib and SRPIN340 synergize the anti-tumor effects in the in vivo model. Nude mice were subcutaneously injected with prostate cancer cell line MDA PCa 2b cells (2×10⁶ cells per nude mouse). After the tumors were visible on nude mice, the mice were intraperitoneal injected every other day with vehicle or Idelalisib (25 mg/kg) in the absence or presence of SRPIN340 (50 mg/kg). The tumors were harvested after drug treatments for 5 weeks. The photograph of tumors harvested from 4 treatment groups were shown in FIG. 13A, and the tumor volumes at the end point (treatment for 5 weeks) were shown as Box-and-Whisker plots in FIG. 13B. The average final volume of the vehicle control group is defined as 100%. Significantly reduced tumor sizes were observed in treatments vs. vehicle control (*p<0.05, ***p<0.005, ****p<0.001, respectively). N=5-6 mice were used in each treatment group.

### High-Throughput Screening for PI3Kδ-S Specific Drugs Using In-Vitro Functional Assays

Small molecule compounds (i.e. from PI3K/AKT/mTOR library, kinase inhibitor library, etc.) were purchased from APExBIO (Huston, TX), and these compounds were used for: treating AA and EA PCa cell lines then subjected to in-vitro functional (i.e. cell viability, proliferation, or apoptosis) assays to evaluate their inhibitory efficacies against AA and EA PCa cells.

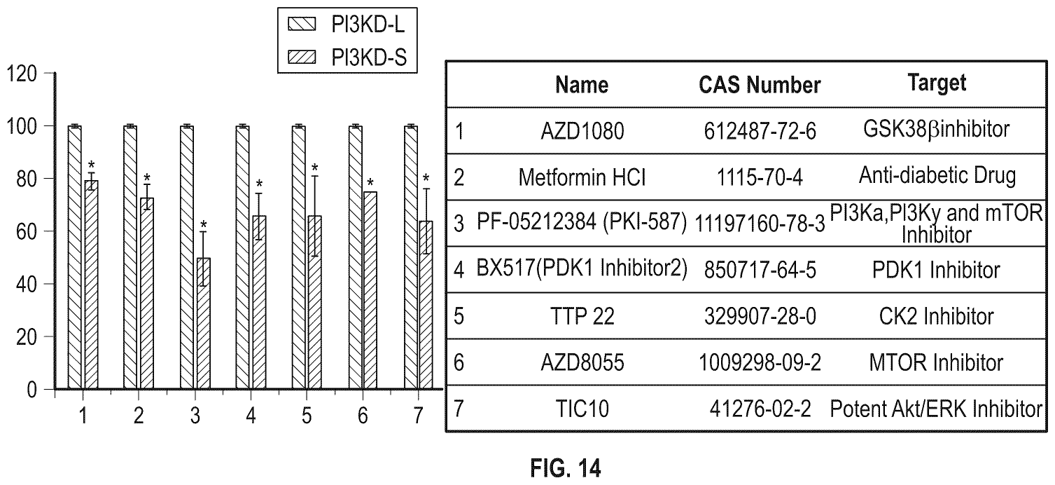

The results evidence that HTS of 178 compounds (from PI3K/Akt/mTOR inhibitor library, APExBIO) using MTT assays in LNCaP cells transfected with PIK3CD-L or PIK3CD-S were performed. Among the 178 compounds, seven compounds were found to inhibit PIK3CD-S expressing (but not PIK3CD-L expressing) PCa cells, as shown in FIG. 14. The same drug treatments in AA PCa cell lines (wild-type AA PCa, and AA PCa transfected with PIK3CD-L or-S) will be conducted for assess the drug efficacies. In vitro HTS using other compound library will be further conducted to identify the effective drugs inhibiting the PCa overexpressing PIK3CD-S.

### Computationally Screening for Small Molecules Targeting PI3Kδ-L/S Isoforms

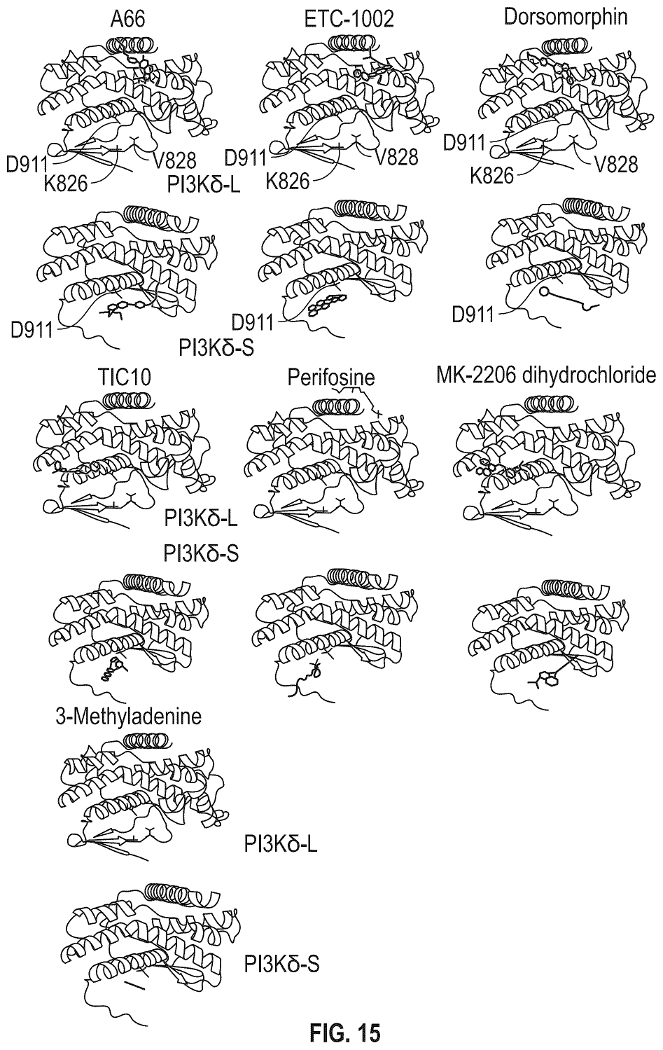

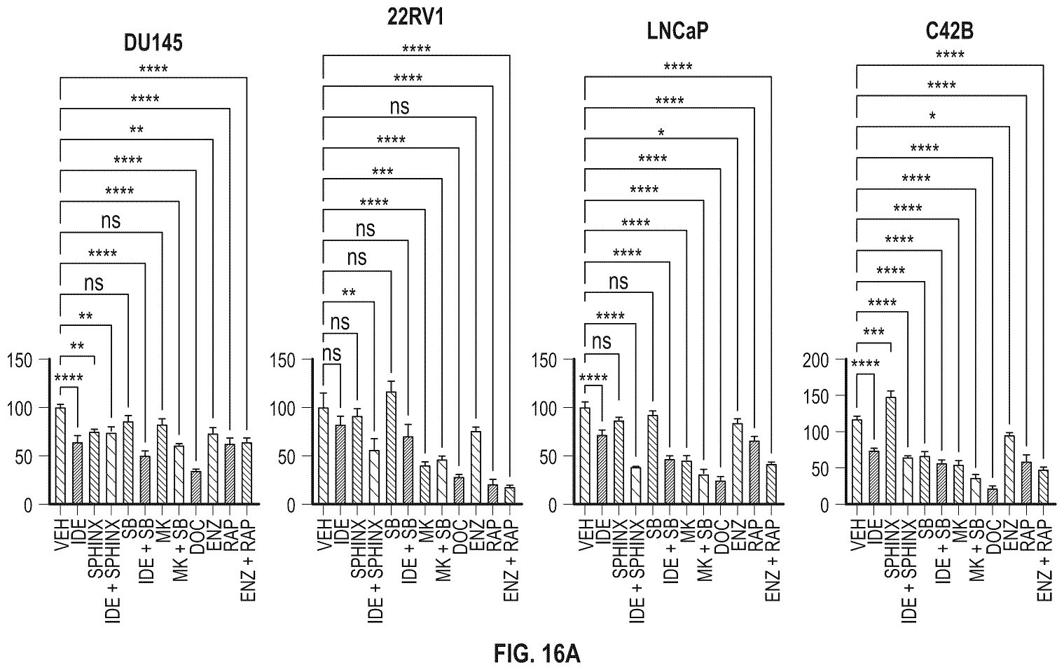

A pilot HTS docking simulation (using AutoDock Vina) simulates the docking of 178 compounds (from PI3K/AKT/mTOR inhibitor library, APExBIO) with PI3Kδ-L and PI3Kδ-S. Among these 178 compounds, the molecular docking results suggested that six compounds (A66, ETC-1002, Dorsomorphin, TIC10, Perifosine, and MK-2206) may specifically target the ATP pocket of the catalytic domain in PI3Kδ-S, but not PI3Kδ-L, as shown in FIG. 15. Intriguingly, 3-Methyladenine (a PI3K Class III inhibitor) was shown to target both PI3Kδ-L and PI3Kδ-S. Ideally, this type of compound will potentially be able to simultaneously target and inhibit PI3Kδ-L and-S, theoretically inhibiting all EA and AA PCa.

Combination Therapies for Treating Prostate Cancer Cell Lines that Express PI3Kδ-L and PI3Kδ-S Splice Isoforms

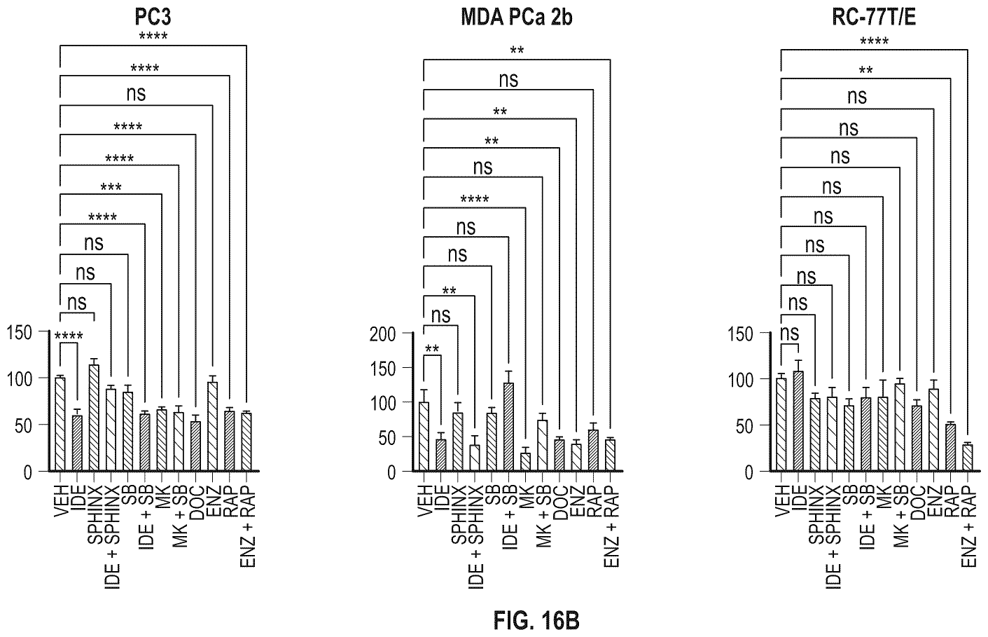

FIGS. 16A-16B show combination therapies for treating prostate cancer cell lines that express PI3Kδ-L and PI3Kδ-S splice isoforms. Seven prostate cancer cell lines (DU145, 22Rv1, LNCaP, C42B, PC3, MDA PCa 2b, and RC-77T/E) were used to test the efficacies of Idelalisib, SPHINX31, MK-2206 2HCl, SB203580, Docetaxel, Enzalutamide, and Rapamycin as single agents and in combinations. The MTT assay results showed that combination therapies using Idelalisib/SPHINX31, Idelalisib/SB203580, MK-2206 2HCl/SB203580, and Enzalutamide/Rapamycin combinations significantly inhibited cancer cell viabilities.

From the foregoing, it can be seen that the present disclosure accomplishes at least all of the stated objectives.

For example, it has been demonstrated PI3Kδ is overexpressed in endocrine cancer or solid tumors in general. PI3Kδ-S splice isoform exhibits a more oncogenic activity (compared to PI3Kδ-L), and is expressed in subgroups of all the cancers that were examined, including PCa, breast, pancreatic, colon and lung cancers. Compared to the full-length PI3Kδ-L, the splice isoform PI3Kδ-S seems to be exempt from the inhibition by PTEN. SRPIN340, a SRPK1/2 inhibitor, reverses the aberrant splicing and sensitizes the advanced endocrine/solid tumors to the PI3Kδ-specific inhibitor, such as Idelalisib. To date, this is the first systemic analysis on the expression profiles of PI3Kδ splice isoforms across different endocrine/solid tumors. The synergistic inhibitory effects of Idelalisib/SRPIN340 combination may pave a new path for developing novel therapeutics for the advanced/refractory endocrine/solid tumors.

### Methods

### Cell Lines and Culture Conditions

PCa (22Rv1, PC-3, LNCaP, MDA PCa 2b, DU-145 and C4-2B), breast (MDA MB 231 and MCF-7), colon (HT-29 and SW620), and lung (A549 and H1299) cancer cell lines were used in this study. All the cancer cell lines were authenticated and purchased from ATCC (Manassas, VA, USA). 22Rv1, LNCaP, H1299 and MCF-7 were cultured in RPMI-1640 with 10% fetal bovine serum (FBS), PC-3 and A549 were cultured in DMEM with 10% FBS, MDA PCa 2b were cultured in BRFF-HPC1 with 20% FBS, DU-145 was cultured in EMEM with 10% FBS, C4-2B was cultured in Advanced DMEM/F12 with 10% FBS, HT-29 was cultured in McCoy's with 10% FBS, SW620 and MDA MB 231 were cultured in L-15 with 10% FBS. Cells were maintained at 37° C. in a 5% CO₂ incubator.

### Tissue Microarrays (TMA)

To perform immunohistochemistry (IHC), different TMAs were used. First, TMAs containing PCa samples and adjacent normal prostate tissues were used to evaluate the PI3Kδ, PI3Kδ-S, PTEN and AMACR expression levels. The TMAs were purchased from US Biomax Inc. (catalog #PR1921b, Derwood, MD, USA). The TMAs contained total of 192 cores, with 80 cases of adenocarcinoma, 8 adjacent normal prostate tissues from PCa and 8 prostate tissues from normal individuals (duplicate cores of each case were printed on this PCa TMA). The pathological features of the cancerous cores were ranging from Gleason Scores of 2+3 to 5+5. To evaluate the expression levels of PI3Kδ, and PI3Kδ-S in various endocrine and solid tumors, TMAs containing tumor samples derived from patients diagnosed with PCa, breast cancer, lung cancer, colon cancer, and pancreas cancer specimens (catalog #BC000119b, US Biomax, Derwood, MD, USA) were used. The TMA contained 38 patient specimens from each of breast cancer, lung squamous cell carcinoma, colon adenocarcinoma, prostate adenocarcinoma and pancreas adenocarcinoma (single core per case). The cores were ranging from grades 1 to 3.

### Immunohistochemistry (IHC) Assays

The protocol for IHC assay was adapted/modified from the previous studies. See e.g., “Gujrati et al., “Downregulation of miR-99b-5p and Upregulation of Nuclear mTOR Cooperatively Promotes the Tumor Aggressiveness and Drug Resistance in African American Prostate Cancer”, Int J Mol Sci, 2022. 23(17); Wang et al., “Identification and Functional Validation of Reciprocal microRNA-mRNA Pairings in African American Prostate Cancer Disparities”, Clin Cancer Res, 2015. 21(21): p. 4970-84. Briefly, TMA slides were first deparaffinized in xylene, followed by immersion in xylene/alcohol (1:1) solution and rehydrated through graded alcohols (100%, 95%, 70% and 50% of alcohol, respectively) to distilled water. Antigen retrieval was performed using EnVision FLEX target retrieval solution from Agilent technologies (Carpinteria, CA, USA). Thereafter, peroxidase block was added dropwise and incubated for 30 min at room temperature. The slides were then washed with 1×PBS twice, blocked with 2.5% BSA/1×PBS for 30 min at room temperature. After discarding blocking buffer, TMAs were incubated with the primary antibody (1:100-1:200 dilutions in 2.5% BSA/1×PBS) at 4° C. overnight. The TMAs were then washed with 1×PBS twice, incubated with HRP-conjugated secondary antibody (Dako, Carpinteria, CA, USA) for 30 min, and the HRP was detected by diaminobenzidine (DAB; Dako, Carpinteria, CA, USA). TMAs were counterstained with Mayer's hematoxylin (Sigma, St. Louis, MO, USA), and mounted with glycergel mounting medium (Dako, Carpinteria, CA, USA). IHC images were captured using Pannormic Midi Digital Scanner (3DHISTECH Ltd., Budapest, Hungary) and visualized using Case Viewer program (3DHISTECH, Budapest, Hungary). The analysis and quantification of IHC images were performed using ImageJ software (NIH, Bethesda, MD, USA), as described in the previous study. The statistical analysis was performed using ANOVA with Tukey's post-hoc test for the multiple comparisons. The PTEN, PI3Kδ, PI3Kδ-S and AMACR antibodies were purchased from Cell Signaling Technology (Waltham, MA, USA), Invitrogen (Waltham, MA, USA) and Agilent Technologies (Santa Clara, CA, USA), respectively.

### Immunofluorescence Assays

First, the 2D monolayer cancer culture was established in a glass bottom dish (Cellvis, CA, USA) with an initial density of 5,000 cells, and the 3D spheroid culture was established in a Nunclon Sphera-treated 96-well plate (catalog #174925, Thermo Fisher Scientific, Waltham, MA, USA) with an initial density of 500-2,000 cells/well according to manufacturer's protocol. After growing for 24 h or 5 days, the cells were washed with 1×PBS and then fixed in 4% paraformaldehyde for 15 min at room temperature. The fixed cells were then permeabilized with 0.1% Triton X-100 for 10 min, and blocked with 2% BSA/1×PBS for 1 h at room temperature. Primary antibodies against PI3Kδ and PI3Kδ-S were added, and the cultures were further incubated overnight at 4° C. Thereafter, the cells were washed three times with 1×PBS, and followed by incubating with Alexa-Fluor-488-conjugated anti-mouse and Alexa-Fluor-594-conjugated anti-rabbit antibodies (Invitrogen, Waltham, MA, USA). After 1 h, the cells were washed three times with 1×PBS and mounted with Prolong glass antifade NucBlue Stain from Invitrogen (cat #P36981, Waltham, MA, USA). The fluorescence signals were visualized using fluorescence microscope (Olympus, MA, USA) or Stellaris confocal microscope (Leica, Deerfield, IL, USA). The fluorescence images of 2D cultures were captured by CellSens V1.18 software (Olympus, Waltham, MA, USA) and analyzed by using ImageJ (NIH, Bethesda, MD, USA). For 3D spheroid cultures, the fluorescence images were captured and processed using Leica Application Suite X (LAS X) software (Leica, Deerfield, IL, USA).

### RT-PCR Assay

The wild-type PCa (22Rv1, PC-3, LNCaP, DU-145, C4-2B, MDA PCa 2b), breast cancer (MDA MB 231 and MCF-7), colon cancer (HT-29 and SW620), and lung cancer (A549 and H1299) cell lines were seeded at a density of 1×10⁵ cells/well in 6 well plates. The cancer cells were cultured at 37° C. in a 5% CO₂ incubator. After culturing the cells for 48 h, the cells were subjected for RNA purification. Selective endocrine/solid tumor cell lines (22Rv1, LNCaP, MDA PCa 2b, HT29, A549 and MCF-7) were grown and then treated with vehicle, 25 μM of Idelalisib, 25 μM of SRPIN340, or combination of Idelalisib (25 μM) and SRPIN340 (25 μM) for 48 h at 37° C. Thereafter, the cells were harvested and subjected to RNA purification. RNA purification was performed using miRNeasy kit from Qiagen (Germantown, MD, USA) according to the manufacturer's protocol. The purified RNA samples were reversely transcribed to cDNA using iScript reverse transcription supermix (Bio-Rad, Hercules, CA). The reverse transcription reactions were performed as follows: 25° C. for 5 min, 46° C. for 50 min, then 95° C. for 1 min. The resulting cDNA samples were used for PCR reactions to examine PIK3CD-L and PIK3CD-S expression profiles, and EIF1AX was used as an endogenous control. The primers used for the PCR reactions are listed in Table 1.

TABLE 1
Primer sequences used for the RT-PCR assays.

| Primer ID | Nucleotide sequences |
|---|---|
| PIK3CD-f | 5′-CTGAGCTCTCAGAAGACC-3′ |
| | (SEQ ID NO: 1) |
| PIK3CD-r1 | 5′-GCTCGCGGTTGATTCCAA-3′ |
| | (SEQ ID NO: 2) |
| PIK3CD-r2 | 5′-AATAGCCAGCACAGGAGAGG-3′ |
| | (SEQ ID NO: 3) |
| EIF1AX-f | 5′-GTACTGGAGAGGGGAGAGCA-3′ |
| | (SEQ ID NO: 4) |
| EIF1AX-r | 5′-TGAAGCTGAGACAAGCAGGA-3′ |
| | (SEQ ID NO: 5) |

### Western Blot Assay

1×10⁶ cells of 22Rv1, LNCaP, MDA PCa 2b, HT-29, A549 and MCF-7 cell lines were seeded in 10-cm plates and the cancer cells were incubated at 37° C. in a 5% CO₂ incubator. After incubation for 24 h, the cells were under different treatments of siRNAs, plasmid or drugs and incubated for additional 48 h. For siRNA knockdown or gene overexpression experiments, the cancer cells were transfected with nonsense/scrambled (NS) RNA, 1 μM of siPIK3CD, 1 μM of siP20 (targeting exon 20 of PIK3CD), 1 μM of siPj (targeting to the junction region between exon 19 and 21 of PIK3CD-S), siPTEN, or pcDNA3-FLAG PTEN. For the drug treatment experiments, the cancer cells were treated with vehicle, 25 μM of Idelalisib, 25 μM of SRPIN340, or a combination of Idelalissib (25 μM) and SRPIN340 (25 μM). After the treatments, the cancer cells were harvested and the protein lysates were extracted using M-PER extraction reagent with protease and phosphatase inhibitor cocktail (Thermo Fisher Scientific, Waltham, MA, USA) according to manufacturer's protocol. Equal amounts of proteins were used based on the quantification using a BCA assay kit (Thermo Fisher Scientific, Waltham, MA, USA), and the proteins were separated by electrophoresis using NuPAGE 4-12% Bis-Tris gels (Invitrogen, Waltham, MA, USA). The gels were transferred to PVDF membranes (Bio-Rad, Hercules, CA, USA) then the PVDF membranes were incubated with primary antibodies and secondary antibodies. The membranes were then incubated with SuperSignal ECL substrates (Thermo Fisher Scientific, Waltham, MA, USA) and the signals were detected using ChemiDoc XRS system (Bio-Rad, Hercules, CA, USA). The primary and secondary antibodies used in the study were mouse monoclonal antibody against PI3Kδ (Santa Cruz Biotechnology, TX, USA), polyclonal antibody against PI3Kδ-S (Invitrogen, Waltham, MA, USA), monoclonal rabbit antibodies against PTEN, pATK, AKT, pS6, S6, β-actin, and anti-rabbit/mouse IgG-HRP antibodies (Cell Signaling Technology, Waltham, MA, USA).

### BrdU-Labeling Cell Proliferation Assay

22Rv1, LNCaP, MDA PCa 2b, HT-29, A549 and MCF-7 cells were seeded at density 5,000 cells/well in 96-well culture plates. The cells were incubated overnight and then were either transfected with NS, siPIK3CD, siPIK3CD-L or siPIK3CD-S. The cells were incubated for another twenty four hours (24 h), then were subjected to bromodeoxyuridine (BrdU) incorporation assay to analyze cell proliferation capacities. The assays were performed using BrdU Cell Proliferation Assay Kit (Sigma-Aldrich, St. Louis, MO, USA) as described by manufacturer's protocol and the previous studies. See e.g., Gujrati et al, “MicroRNA-mRNA regulatory network mediates activation of mTOR and VEGF signaling in African American prostate cancer.” Int J Mol Sci (2022) 23 (6), which is hereby incorporated by reference in its entirety herein. The measurements were based on the absorbances at dual wavelengths of 450 nm and 540 nm using Multiskan FC microplate photometer (Thermo Scientific, Waltham, MA, USA).

### MTT Assays of the Spheroid Cultures Under Drug Treatments

The endocrine/solid tumor cell lines 22Rv1, LNCaP, MDA PCa 2b, HT29, A549 and MCF-7 were seeded at densities of 500-2,000 cells/well in the 96-well Nunclon Sphera-treated plates (catalog #174925, Thermo Fisher Scientific, Waltham, MA, USA) containing DMEM/10% FBS media. The tumor spheroids were first incubated at 37° C. in a 5% CO₂ incubator for 2 days, then 25 μM of Idelalisib, 25 μM of SRPIN 340, or a combination of Idelalisin (25 μM) and SRPIN340 (25 μM) were added as treatments for additional 5 days. For monitoring the spheroid growths, each well was imaged every day using Olympus IX73 microscope (Olympus, Bartlett, TN, USA) and then the spheroid diameter, area and circularity were measured by ImageJ. For measuring the cell viabilities of spheroids under different drug treatments, the CellTiter 96 Non-Radioactive Cell Proliferation Assay reagent (Promega, Madison, WI, USA) was added to each well and incubated with the spheroids for 3 h at 37° C., then the solubilization solution was added. After incubation for 1 h, the samples were detected by the Multiskan FC microplate photometer (Thermos Fisher Scientific, Waltham, MA, USA) at the wavelength of 570 nm. The data were analyzed by GraphPad Prism 9 program (GraphPad, La Jolla, CA, USA).

### Survival Curves for Cancer Patients Expressing High and Low Levels of PI3Kδ

The survival curves showing the changes of survival rates along the time in cancer patients expressing high and low levels of PI3Kδ were plotted using PanCanSurvPlot program (https://smuonco.shinyapps.io/PanCanSurvPlot/). PanCanSurvPlot retrieves microarray or RNA-sequencing data of cancer patients from the GEO and TCGA databases, and further performs the survival analysis. The program collected a total of 215 cancer-related databases from the GEO and TCGA databases, covering ˜45,000 samples from 51 different cancer types and 13 survival outcome datasets. The survival data analyses were performed using Kaplan-Meier method, and long-rank test and univariant Cox proportional hazard regression model was utilized to assess the correlation between gene expression profiles and clinical outcomes. The end users can define the patient groups with high and low expression levels of specific gene, based on the median or optimal cutoff values. In FIG. 12, patient cohorts were selected from PCa, breast, pancreatic, ovarian, endometrial, cervical, colon and lung cancers, and divided cancer patient groups with high and low PI3Kδ expression levels based on ‘optimal’ cutoff values. The survival curves showing significantly lower survival rates (p-values<0.05, long-rank test and univariant Cox proportional hazard regression) in patients expressing high PI3Kδ vs. low PI3Kδ were plotted.

### Quantification of PI3Kδ-L and PI3Kδ-S Expression Levels in PCa and Other Endocrine/Solid Tumor Cell Lines

The percentage of PI3Kδ and PI3Kδ-S fluorescence-positive cells were counted by measuring (numbers of green signals/numbers of blue signals×100%) and (numbers of red signals/numbers of blue signals×100%) signals from the immunofluorescence assay results in FIG. 1D and FIG. 2D. PI3Kδ and PI3Kδ-S fluorescence intensities of each images from immunofluorescence assays in FIG. 1D and FIG. 2D were quantified using ImageJ, as described in Methods. In each cell line, PI3Kδ fluorescence intensity was defined as 100%. PI3Kδ-S intensity was quantified and normalized to the PI3Kδ fluorescence intensity in each cell line. The PI3Kδ-L intensities were calculated using equation of: 100%−(% of PI3Kδ-S).

TABLE 2
Quantification of PI3Kδ-L and PI3Kδ-S expression levels in PCa (A) and other
endocrine/solid tumor cell lines (B).
A.

| | | | | | | MDA |
|---|---|---|---|---|---|---|
| | 22Rv1 | PC-3 | LNCaP | DU-145 | C42B | PCa 2b |
| PI3Kδ (+) cells, % | 100 | 100 | 100 | 100 | 100 | 100 |
| PI3Kδ-S (+) cells, % | 100 | 100 | 33 | 100 | 100 | 100 |
| PI3Kδ intensity, % | 100 | 100 | 100 | 100 | 100 | 100 |
| PI3Kδ-S intensity, % | 59 | 49 | 11 | 39 | 24 | 72 |
| PI3Kδ-L intensity, % | 41 | 51 | 89 | 61 | 76 | 28 |
| PI3Kδ-S:PI3Kδ | 3:5 | 1:2 | 1:9 | 1:2.5 | 1:4.2 | 7.2:10 |
| PI3Kδ-S/PI3Kδ-L ratio | 1.44 | 0.96 | 0.12 | 0.64 | 0.32 | 2.6 |
| (S/L ratio) | | | | | | |

B.

| | | | | | MDA | |
|---|---|---|---|---|---|---|
| | HT-29 | SW620 | A549 | H1299 | MB 231 | MCF-7 |
| PI3Kδ (+) cells, % | 100 | 100 | 100 | 100 | 100 | 100 |
| PI3Kδ-S (+) cells, % | 100 | 100 | 100 | 100 | 100 | 50 |
| PI3Kδ intensity, % | 100 | 100 | 100 | 100 | 100 | 100 |
| PI3Kδ-S intensity, % | 76 | 74 | 29 | 12 | 30 | 41 |
| PI3Kδ-L intensity, % | 24 | 26 | 71 | 88 | 70 | 59 |
| PI3Kδ-S:PI3Kδ | 1:1.3 | 0.75:1 | 1:3.5 | 1:10 | 1:3 | 1:2.5 |
| PI3Kδ-S/PI3Kδ-L ratio | 3.17 | 2.85 | 0.41 | 0.14 | 0.43 | 0.70 |
| (S/L ratio) | | | | | | |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A therapeutic comprising:
a phosphoinositide 3-kinase (PI3K) inhibitor; and
a serine-arginine-rich protein kinase (SRPK) inhibitor.

2. The therapeutic of claim 1, wherein the PI3K inhibitor comprises a phosphoinositide 3-kinase-8 (PI3Kδ) inhibitor.

3. The therapeutic of claim 2, wherein the PI3Kδ inhibitor comprises idelalisib.

4. The therapeutic of claim 1, wherein the SRPK inhibitor comprises SRPIN340.

5. The therapeutic of claim 1, wherein the SRPK inhibitor comprises a SRPK1/2 inhibitor.

6. The therapeutic of claim 5, wherein the SRPK1/2 inhibitor comprises SRPKIN-1 or SPHINX31.

7. The therapeutic of claim 1, further comprising an additional tumor suppressor.

8. The therapeutic of claim 7, wherein the additional tumor suppressor comprises phosphatase and tensin homolog (PTEN).

9. A method comprising:
reversing an aberrant splicing in tumor cells with a serine-arginine-rich protein kinase (SRPK) inhibitor.

10. The method of claim 9, further comprising, prior to the reversing step, using a phosphoinositide 3-kinase-δ (PI3Kδ) inhibitor to target tumor cells.

11. The method of claim 9, further comprising further sensitizing the tumors cells to the PI3Kδ inhibitor.

12. The method of claim 9, further comprising converting PI3Kδ-S to PI3Kδ-L.

13. The method of claim 9, further comprising inhibiting PI3Kδ-S synthesis.

14. The method of claim 9, further comprising RNA splice switching.

15. The method of claim 9, further comprising utilizing phosphatase and tensin homolog (PTEN) to further regulate PI3K/AKT/mTOR signaling.

16. The method of claim 9, wherein a type of the tumor cells is selected from the group consisting of: PCa, breast, endocrine, pancreatic, colon, and lung cancers.

17. A method of biological identification, the method comprising:
identifying whether there is presence of a precision biomarker comprising PI3Kδ-L or PI3Kδ-S.

18. The method of claim 17, further comprising diagnosing cancer or predicting an outcome of a disease based upon the presence of the precision biomarker.

19. The method of claim 17, further comprising measuring an aspect of the AKT/mTOR signaling pathway after confirming the presence of the precision biomarker.

20. The method of claim 17, further comprising measuring a biological state or a condition in a solid tumor.


SEQUENCE LISTING (5 sequences)

SEQ ID NO: 1 - DNA, 18 bp, synthetic construct
  source 1..18: mol_type = other DNA
ctgagctctcagaagacc

SEQ ID NO: 2 - DNA, 18 bp, synthetic construct
  source 1..18: mol_type = other DNA
gctcgcggttgattccaa

SEQ ID NO: 3 - DNA, 20 bp, synthetic construct
  source 1..20: mol_type = other DNA
aatagccagcacaggagagg

SEQ ID NO: 4 - DNA, 20 bp, synthetic construct
  source 1..20: mol_type = other DNA
gtactggagaggggagagca

SEQ ID NO: 5 - DNA, 20 bp, synthetic construct
  source 1..20: mol_type = other DNA
tgaagctgagacaagcagga